(19) United States
(12) Patent Application Publication
(10) Pub. No.: US 2012/0135039 A1
(43) Pub. Date: May 31, 2012

(54) Oral Vaccines for Producing Mucosal Immunity

(76) Inventors: Frank E. Aldwell, Dunedin (NZ); Kenneth W. Beagley, Moggill (AU)

(21) Appl. No.: 13/119,493

(22) PCT Filed: Oct. 7, 2009
(86) PCT No.: PCT/IB2009/007232

§ 371 Date: May 23, 2011

Related U.S. Application Data
(60) Provisional application No. 61/195,631, filed on Oct. 8, 2008.
(60) Provisional application No. 61/195,882, filed on Oct. 10, 2008.

(51) Int. Cl. A61K 39/118; A61P 37/04; A61P 31/04; A61K 39/02

(52) U.S. Cl. 424/263.1; 424/234.1

(57) ABSTRACT

Embodiments of this invention include lipid-based immunogenic compositions (adjuvants or carriers) useful for oral or gastrointestinal administration for improving mucosal immune responses in animals vaccinated for a variety of bacterial infections. In certain embodiments, lipid compositions of this invention include a mixture of fatty acids having different chain lengths, thereby providing desired physico-chemical properties. When a bacterial antigen is mixed with a lipid-based adjuvant or carrier, the resulting composition elicits improved mucosal immune responses and thereby decreases infections and sequellae of disease caused by Chlamydia or Helicobacter.

### CLAIM OF PRIORITY

This application is filed under 35 U.S.C. §371 as a National Phase application based on PCT International Patent Application, which claims priority to U.S. Provisional Patent Application No. 61/195,631 filed 8 Oct. 2008, entitled “Immunogenic Compositions,” Frank E. Aldwelt and Kenneth W. Beagley, inventors, and to U.S. Provisional Patent Application No. 61/295,882 filed 10 Oct. 2008, entitled “Adjuvants for Immunogenic Responses,” Frank E. Aldwell and Kenneth W. Beagley, inventors. All of these applications are herein incorporated fully by reference.

### FIELD OF THE INVENTION

This invention relates generally to compositions suitable for storing, administering and improving the immunogenicity of antigens or immunogens used in vaccines. Particularly, this invention relates to lipid-based adjuvants or carriers useful for improving immune responses to bacterial antigens. More particularly, this invention relates to lipid-based adjuvants or carriers having specific lipid components, and uses thereof to provide improved immune responses to infections caused by Chlamydia and Helicobacter.

### BACKGROUND

A large number of infectious pathogens invade mucosal surfaces resulting in infection and disease. Two important mucosal pathogens affecting both human and animal populations worldwide are Chlamydia and Helicobacter. The World Health organization (WHO) estimated that in 1999 there were 92 million new cases of Chlamydia genital infections worldwide and the incidence of infection continues to increase in both developed and developing countries (K W, Timms P. Journal of Reproductive Immunology 2000; 48(1):47-68). Helicobacter is believed to infect 50% of the world's population, with rates exceeding 90% in some developing countries
(Del Giudice, G., et al., Annu Rev Immunol, 2001. 19: p. 523-63; Frenck, R. W., Jr. and J. Clemens, Microbes Infect, 2003. 5(8): p. 705-13).

Chlamydia

Members of the genus Chlamydia cause a plethora of ocular, genital and respiratory diseases, with severe complications, such as blinding trachoma, pelvic inflammatory disease, ectopic pregnancy and tubal factor infertility, interstitial pneumonia, and chronic diseases that may include atherosclerosis, multiple sclerosis, adult-onset asthma and Alzheimer's disease.

Chlamydia trachomatis and C. pneumoniae infects a variety of mucosal surfaces causing a number of diseases including pelvic inflammatory disease (PID), infertility, trachoma resulting in blindness, respiratory disease, atherosclerosis and exacerbations of asthma (Faal, N., et al., PLoS Med, 2006. 3(8): p. e266; Mabey, D. and R. Peeling, Sexually transmitted infections, 2002. 78(2): p. 90-2; Hansbro, P. M., et al., Pharmacol Ther, 2004. 101(3); p. 193-210; Horvat, J., et al., Am J Respir Crit. Care Med, 2007).

Organisms of the family Chlamydiae are obligate intracellular bacteria. They lack several metabolic and biosynthetic pathways and depend on the host cell for intermediates, including ATP. Chlamydiae exist as two stages: (1) infectious particles called elementary bodies and (2) intracytoplasmic, reproductive forms called reticulate bodies. There are three described species of Chlamydia that commonly infect humans. C. trachomatis causes the eye disease Trachoma and the sexually transmitted infection, Chlamyidia. C. psittaci causes psittacosis and C. pneumoniae causes a form of pneumonia. Additionally, mice are susceptible to C. muridarum, which causes infections of the murine reproductive tract. The first two contain many serovars based on differences in cell wall and outer membrane proteins. Chlamydia pneumoniae contains one serovar—the TWAR organism.

Chlamydiae have a hemagglutinin that may facilitate attachment to cells. The cell-mediated immune response is largely responsible for tissue damage during inflammation, although an endotoxin-like toxin has been described.

Most human and animal pathogens including Chlamydia initiate infection via mucosal surfaces. Similarly, genital infections with Chlamydia may arise from infection of mucosal surfaces. Accordingly, protective immunity against such pathogens may require induction of strong mucosal immune responses. Despite the obvious need for vaccines to protect against infections via mucosal sites, the vaccines in use today are given by intradermal or subcutaneous injection. However, mucosal immune responses are generally weak following parenteral immunisation.

Chlamydia trachomatis infections are the most common sexually transmitted bacterial infections worldwide. Chlamydia trachomatis causes sexually transmitted genital and rectal infections. The frequency of C trachomatis infections in men may equal or exceed the frequency of gonorrhea. Nongonoccocal urethritis, epididymitis, and proctitis in men can result from infection with C trachomatis. Superinfection of gonorrhea patients with C trachomatis also occurs. Acute salpingitis and cervicitis in young women can be caused by a C trachomatis infection ascending from the cervix. A high rate genital tract co-infection by C trachomatis in women with gonorrhea has been reported. Chlamydia trachomatis was isolated from the fallopian tubes of infected women. In one report C trachomatis elementary bodies attached to spermatozoa were recovered from the peritoneal cavity of patients with salpingitis.

Neonates exposed to C trachomatis in an infected birth canal may develop acute conjunctivitis within 5 to 14 days. The disease is characterized by marked conjunctival erythema, lymphoreticular proliferation, and purulent discharge. Untreated infections can develop into pneumonitis; this type of pneumonitis occurs only during the first 4 to 6 months of life.

Recently, C trachomatis has been suspected of causing lower respiratory tract infections in adults, and several cases of C trachomatis pneumonia have been reported in immunocompromised patients from whom the pathogen was isolated. Evidence also indicates that C trachomatis may cause pneumonia or bronchopulmonary infections in immunocompetent persons. Sequellae associated with C. trachomatis infections include pelvic inflammatory disease, ectopic pregnancy and infertility, the most costly health outcomes of any STI except HIV/AIDS (Westrom L, Mardh P. A., Br Med Bull 1983 April; 39(2):145-50). Furthermore, an existing chlamydial infection increases the risk of contracting HIV (Ho J L, et al., J Exp Med 1995 Apr. 1; 181(4):1493-505) and Herpes simplex infections (Kaul R, et al., J Infect Dis 2007 Dec. 1; 196(11):1692-7). Due to the asymptomatic nature of most chlamydial infections (Stamm W E. In: Woodall J P, editor. Proceedings of the Chlamydia Vaccine Development Colloquium; 2004; Alexandria, Va.: The Albert B. Sabin Vaccine Institute; 2004. p. 15-8), the availability of effective antibiotic treatment has not been able to slow the increasing incidence of infection and it is generally believed that an effective vaccine is required control this silent epidemic.

### Helicobacter

Bacteria of the genus Helicobacter, including H pylori are considered important causes of several types of gastrointestinal diseases. Helicobacter infection of the gastric mucosae is associated with the development of diseases such as chronic active gastritis, gastric ulcers, duodenal ulcers and is associated with the development of gastric adenocarcinoma (Enno, A., et al., Am J Pathol, 1998. 152(6): p. 1625-32; Correa, P., J Natl Cancer Inst, 2003. 95(7): p. E3; Ernst, P. and B. Gold, Annu Rev Microbiol, 2000. 54: p. 615-40; Uemura, N., et al., N Engl J Med, 2001. 345(11): p. 784-9).

### SUMMARY

We have discovered previously unappreciated problems in the art, namely, that although many human and animal pathogens infect the organism via the mucosae, development of effective vaccines that act in the mucosae to protect the animals has been very difficult if not impossible. A major problem exists with the degradation of orally delivered vaccine antigens due to gastric acidity and proteolytic destruction before they reach immune inductive sites such as Peyer's patches. Inadequate stimulation of gut-associated lymphoid tissues can also induce oral tolerance rather than adaptive immunity. Further, even though rodent immunity can be increased with the use of cholera toxin (CT), CT is not tolerated by human beings. No efficacious vaccines for either Chlamydia or Helicobacter have been approved for use in humans. Thus, there is now a great need for compositions and methods that are effective at the mucosae and mimic the beneficial effects of cholera toxin, but without the harmful side effects observed in human beings.

We have unexpectedly discovered that certain lipid compositions, especially those containing long-chain fatty acids, when used as adjuvants or carriers, can solve these and other problems to promote mucosal immunity and provide protection against mucosal infections caused by Chlamydia and Helicobacter.

We have also unexpectedly found that certain lipid compositions when used as adjuvants or carriers along with isolated Chlamydia antigens can provide immunity against Chlamydia that is as effective as cholera toxin, but without the detrimental toxic side effects. This finding was completely unexpected based on prior observations that certain lipid compositions can improve immune responses when used with living organisms (PCT/NZ2002/00132) U.S. Pat. No. 7,758,869, issued Jul. 20, 2010 incorporated herein fully by reference. Similarly, we have unexpectedly found that certain lipid compositions when used as adjuvants or carriers along with H pylori antigens can provide immunity against mucosal H pylori infections. This finding was completely unexpected based on prior observations.

Additionally, a major problem exists with oral vaccination due to degradation of vaccine antigens in the stomach and other parts of the digestions system. These problems may be due to gastric acidity and/or proteolytic destruction of the antigens before they reach immune inductive sites such as Peyer's patches. Thus, inadequate stimulation of gut-associated lymphoid tissues (“GALT”) can also induce oral immune tolerance rather than adaptive immunity, leading not to protection but can exacerbation of disorders associated with antigens.

Thus, this disclosure presents the first demonstration that a killed antigen (as opposed to live or attenuated organism) can be administered in an orally active vaccine, and can induce mucosal immunity, thereby protecting the animal from mucosal infections by pathogenic organisms.

### BRIEF DESCRIPTION OF THE FIGURES

This invention is described with reference to specific embodiments thereof. Other features of this invention can be appreciated by reference to the figures, in which:

FIGS. 1A and 1B depict graphs of MOMP-specific antibodies in serum (FIG. 1A) and vaginal lavage (FIG. 1B) IgG and IgA was determined by ELISA. The y axis shows the ratio of the endpoint titer (E.P.T) determined by the division of the immunization group E.P.T by the non-immunized control E.P.T. Lipid C and Chlamydia MOMP together produced about a doubling of production of IgA, compared to non-immunized animals, an effect that was similar to that observed with CpG/CT and MOMP together. We conclude that lipid C can increase the immunological response of the vaginal mucosa to Chlamydia MOMP antigen. Results are representative of two separate experiments containing 5 mice per group in each experiment. * p<0.05, ** p<0.01, compared to non immunized control. Error bars, standard error of mean.

FIGS. 2A and 2B depict graphs of bacterial recovery from vaginal swabs following live bacterial challenge with C. muridarum. Vaginal swabs were collected at 3-day intervals from immunized (MOMP mixed with CpG/CT, Lipid C formulated MOMP or Lipid C formulated MOMP mixed with CpG/CT) and control mice following intravaginal challenge with C. muridarum. Live bacterial recovery (bacterial shedding) from vaginal swabs was determined via cell culture at 3-day intervals (FIG. 2A). The total level of infectivity was determined by measuring the area under each curve (FIG. 2B). Results are representative of two separate experiments of 5 animals in each group. We found unexpectedly that lipid C and MOMP together decreased bacterial shedding by about 60% compared to animals exposed to MOMP alone (FIG. 2B). CpG/CT and MOMP together decreased bacterial shedding by 57%. Furthermore, we unexpectedly found that lipid C decreased bacterial shedding (lipid C+MOMP+CpG/CT) by about 48% compared to animals treated with CpG/CT+MOMP * p<0.05, *** p<0.001 compared to non immunized control. Error bars, Standard error of mean. These results indicate that lipid C can act synergistically with CpG/CT to increase immunological responses of the vaginal mucosa.

FIGS. 3A and 3B depict graphs of H pylori specific-antibodies in serum (top) and fecal pellet wash (bottom). IgG and IgA was determined by ELISA. The y axis shows the ratio the endpoint titer (E.P.T) determined by the division of the immunization group E.P.T by the non immunized control E.P.T. Results are representative of two separate experiments containing 5 mice per group in each experiment. FIG. 3A shows that Lipid C increased production of H pylori-specific IgG in the Serum compared to non-immunized controls. Further, FIG. 3B shows that lipid C increased H pylori-specific IgA in the fecal pellets (FIG. 3B). The degree of protection observed with lipid C and H pylori antigen was about 25% reduction in bacteria recovered, which represents a decreased bacterial load in the organism. Error bars, Standard error of mean. These results indicate that lipid C can promote mucosal immunity in the gastrointestinal tract.

FIGS. 4A, 4B, and 4C depict graphs of Chlamydia MOMP-specific antibodies in serum (FIG. 4A), bronchoalveolar lavage BAL (FIG. 4B) and vaginal lavage (FIG. 4C). IgG and IgA titers were determined by ELISA. The y axis shows the ratio of the endpoint titer (E.P.T) determined by the division of the immunization group E.P.T by the non immunized control E.P.T. Results are representative of two separate experiments containing 5 mice per group in each experiment. * p<0.05, ** p<0.01, compared to non immunized control. Error bars, standard error of mean. Oral lipid C plus MOMP produced a modest increase in serum IgG (FIG. 4A). Oral lipid C plus MOMP had little effect in the respiratory tract (FIG. 4B). In contrast, oral lipid C plus MOMP increased vaginal MOMP-specific IgA by about 2-fold (FIG. 4C).

FIG. 5 depicts a graph of bacterial recovery following intragastric challenge with H pylori SSI. Mice were challenged one week after final immunization with two intragastric inoculations of 1×10⁷ cfu's of Helicobacter pylori SS1. At 6 weeks following live bacterial challenge stomach tissue was homogenized and cultured for 6 days on CSA agar plates containing GLAXO-supplement (see materials and methods). The y-axis shows the number colony forming units/gram (cfu/gram) of homogenized stomach tissue, represented by a log scale. Immunization with the combination of lipid C and H pylori SSI antigen resulted in about a 25% reduction in bacteria recovered from stomach tissue six weeks following intragastric inoculation compared to non-immunized animals. H Pylori antigen plus CpG/CT decreased bacterial recovery, and addition of lipid C further decreased bacterial recovery by about 85% compared to non-immunized controls. Results are representative of two separate experiments. * p<0.05 compared to non immunized control; n=5 animals in each group per experiment. Error bars, Standard Error of Mean. These results indicate that oral compositions containing lipid C can promote mucosal immunity against H pylori in the stomach.

FIG. 6 depicts a graph of bacterial recovery from lung tissue following live bacterial challenge with C. muridarum. Female BALB/c TCI orally immunized with MOMP mixed with CpG/CT, lipid C formulated MOMP or lipid C formulated MOMP mixed with CpG/CT and non-immunized control mice were challenged intra-nasally with live C. muridarum. Lungs were removed 12 days after bacterial challenge (peak infection point) and the amount of live Chlamydia recovered was determined by culture. Results are representative of two separate experiments. These results show that oral immunization with lipid C and MOMP together, decrease the recovery of Chlamydia compared to non-immunized controls * p<0.05; n=5 animals in each group per experiment, error bars are Standard Error of Mean.

### DETAILED DESCRIPTION

### Mucosal Immunity

Mucosal surfaces are a major portal for the entry of pathogenic organisms. As such, they are defended by a mucosal immune system that is functionally and anatomically distinct from the systemic immune system. The mucosal and systemic immune systems work in conjunction to provide protection against pathogens. In the intestines, antigen presenting cells (“APCs”) sample luminal antigens presenting epitopes to lymphocytes within Peyer's patches and draining mesenteric lymph nodes (Owen, R. and A. Jones, Gastroenterology, 1974. 66(2): p. 189-203; Iwasaki, A. and B. Kelsall, J Exp Med, 2000. 191(8): p. 1381-94). As a major mucosal inductive site abundant in both APCs and lymphocyte populations, gut-associated lymphoid tissue (“GALT”) represents an attractive site for induction of protective mucosal immunity through oral immunization.

The gastrointestinal tract is exposed to a variety of antigens, which includes ‘self’ antigens generated from normal metabolic processes, ingested food antigens and those from commensal flora or pathogenic organisms. To function effectively, the immune system is required to differentiate between ‘good’ antigens from those that maybe ‘harmful’ to host. Oral tolerance is the specific mechanism by which the immune system generates a state of immune unresponsiveness against those antigens deemed non harmful. The uptake of antigens for immune presentation following oral immunization can be achieved through a number of mechanisms. Enterocytes take up, process and present antigen to T cells on basolaterally expressed MHC class II molecules. (S. G. Mayrhofer, and L. Spargo, in Immunology. 1990; Hershberg, R. M., et al., J Clin Invest. 1998. p. 792-803).

Dendritic cells (“DCs”) located throughout the lamina propria sample luminal antigens by ‘squeezing’ their dendrites through tight junctions between epithelial cells (Rescigno, M., et al., Nat Immunol, 2001. 2(4): p. 361-7). Microfold (“M”) cells non-specifically transport luminal antigens across intestinal epithelial barrier to underlying antigen presenting cells (“APCs”) including DCs and macrophages (Bockman, D., et al., Ann N Y Acad Sci, 1983. 409: p. 129-44; Bockman, D. and M. Cooper, Am J Anat, 1973. 136(4): p. 455-77; Neutra, M. R., et al., Cell Tissue Res. 1987. p. 537-46). Protein antigens introduced to the GALT through active immunization generally results in the induction of tolerance rather then immunity.

Because the gastrointestinal tract is a portal and because it forms an easily accessible, non-invasive route for vaccination, oral immunization has long been viewed as an attractive means of protecting the host against infectious agents that invade the body across the mucosal surfaces lining the gastrointestinal, respiratory and urogenital tracts. The potential of oral immunization, as demonstrated in many animal studies, has not been realized in humans, with only the oral polio, oral typhoid and oral cholera vaccines approved for human use, all of which are live attenuated vaccines. Limitations that have prevented the use of oral immunization in humans include the need for large antigen doses, destruction of antigen by normal digestive processes and the need for strong mucosal adjuvants to overcome the induction of oral tolerance that is often induced by feeding of protein subunit antigens (Weiner H L. J Clin Invest 2000 October; 106 (8):935-7).

Oral immunization is a needle-free, cost effective method that is easy to administer and is not associated with the risk of spreading diseases from person to person such as HIV, Hepatitis B and Hepatitis C (Giudice, E. L. and J. D. Campbell, Adv Drug Deliv Rev. 2006. p. 68-89). The oral route is also an important method for the immunization of wild animals. Oral regimes avoid the stress of animals that is associated with currently used invasive capture and release disease management methods (Cross, M. L. et al., Vet J. 2006). For these reasons the oral route provides the potential for immunization of both large animal and human populations in order to minimize the spread of communicable diseases. Commercial oral vaccines widely used in humans include the Sabin polio vaccine, the live-attenuated typhoid vaccine and the killed whole-cell B subunit and live attenuated cholera vaccines.

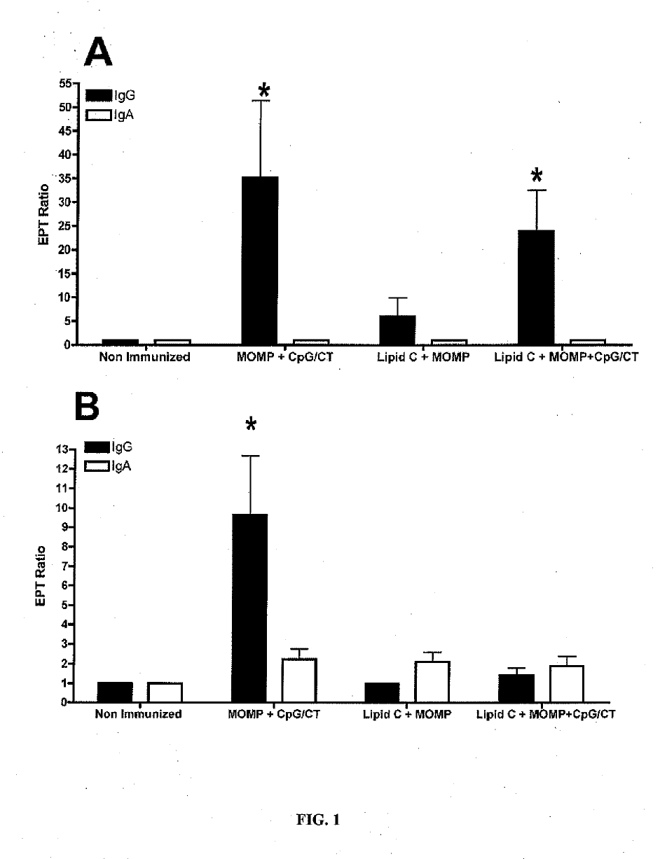

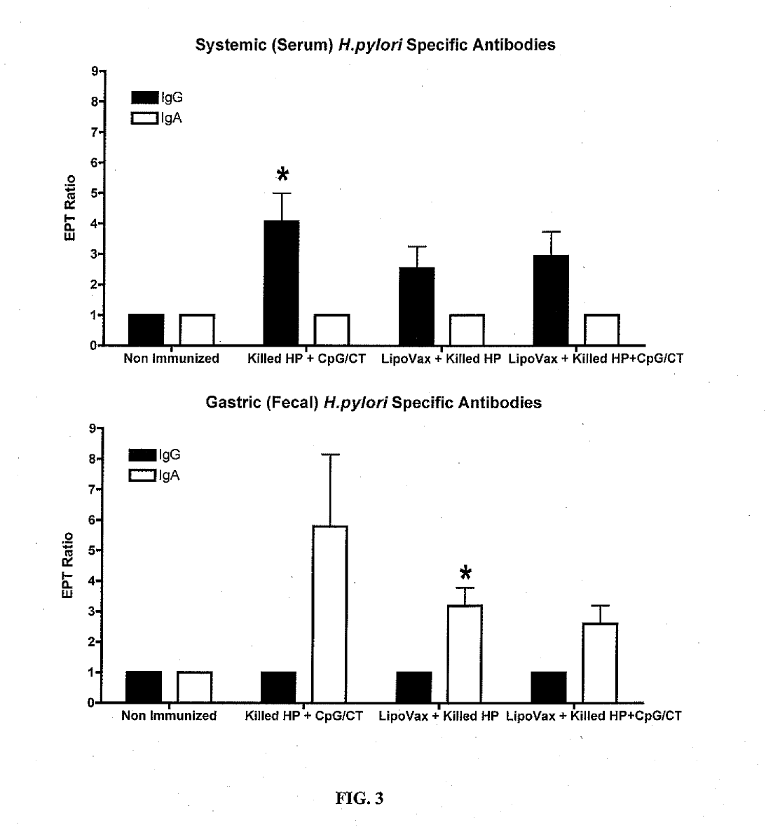

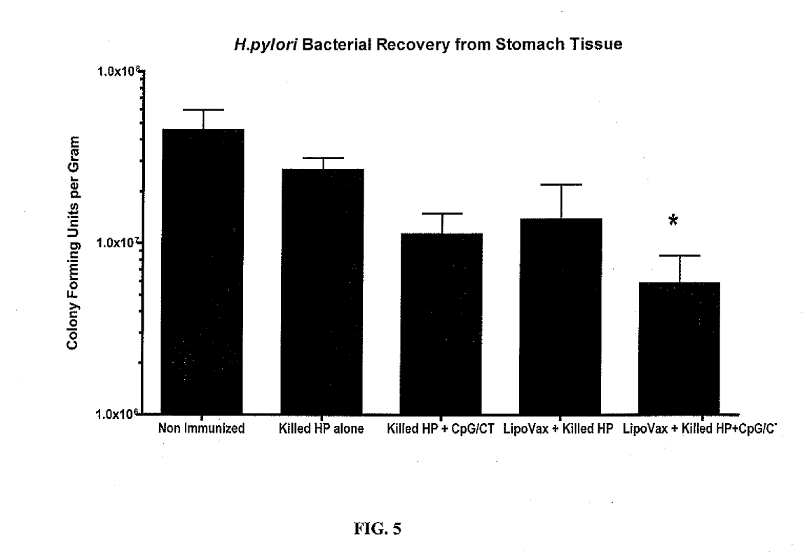

These results show that vaccines of this invention given orally can act at the stomach (FIGS. 3 and 5) in the case of H pylori and at the genital tract and lung in the case of Chlymdia (FIGS. 1, 2, 4, and 6). These results also show that delivery of a vaccine of this invention to a mucosal surface is capable of triggering responses at a variety of other mucosal surfaces. These findings therefore demonstrate that vaccine compositions of this invention provide solutions to long-standing problems in the art. The findings that lipid compositions of this invention can further increase mucosal immunity caused by traditional adjuvants CpG and CT, indicates that the compositions of this invention can act synergistically to promote mucosal immunity.

### Vaccine Adjuvants

To improve immune responses, antigens have been mixed with a number of adjuvant substances to stimulate immunogenicity. Commonly used adjuvants include alum and oil-in-water emulsions. The latter group is typified by the Freund's mineral oil adjuvants. However, the use of Freund's complete adjuvant (“FCA”) in human and veterinary vaccines is contraindicated because of toxic reactions that have been reported. For these reasons, Freund's adjuvant may also be unsuitable for oral administration.

In oil-in-water emulsions surfactants have been required because of the high oil content. Detergent properties of surfactants have rendered them unsuitable for parenteral or oral administration. Further, toxic reactions even for approved surfactants have been reported. Further drawbacks with emulsions are that they are heterogeneous systems of one immiscible liquid dispersed in another. This preparation is often unstable and results in separation of the aqueous phase over time, and therefore poses difficulties for maintaining vaccines in stable suspension. Moreover, antigens trapped in the aqueous phase of water-in-oil emulsions or traditional liposomes are unlikely to be protected from degradation in the stomach or other portions of the digestive system. In contrast, lipid-containing compositions of this invention and methods for their use can protect fragile protein antigens in the digestive tract, thereby permitting them to have access to Peyer's patches and other immunologically sensitive structures within the gastrointestinal tract, and thereby provide immunological protection to the mucosae.

### ADP-Ribosylating Exotoxins

To enhance adaptive immunity following oral immunization with poorly immunogenic protein antigens, adjuvants such as ADP-ribosylating exotoxins (“bAREs”) are used to enhance immune activation and prevent the induction of oral tolerance. The most commonly used adjuvants in animal studies of oral immunization, the ADP-ribosylating bacterial exotoxins (ABARES) (Williams N A, Hirst T R, Nashar T O. Immunol Today 1999 February; 20(2):95-101) such as cholera toxin (“CT”) and E. coli heat labile toxin (“LT”), cannot be used in humans because of both gastric and neurological toxicity (van Ginkel F W et al., J Immunol 2000; 165(9):4778-82). Because of this the potential of oral immunization in humans will only be realized if safe adjuvants can be found to replace adjuvants such as ABARES. ABAREs such as CT or LT are potent stimulators of mucosal immunity and have been used experimentally in a number of immunization routes including oral, intranasal and transcutaneous (Holmgren, J., et al., Vaccine, 1993. 11(12): p. 1179-84; Hickey, D. K., et al., Vaccine, 2004. 22(31-32): p. 4306-15; Skelding, K. A., et al., Vaccine, 2006. 24(3): p. 355-66; Glenn, G., et al., J Immunol, 1998. 161(7): p. 3211-4; Yu, J., et al., Infect Immun, 2002. 70(3): p. 1056-68; Berry, L. J., et al., Infect Immun, 2004. 72(2): p. 1019-28). However, their use for veterinary and human immunization regimes using the oral and intranasal route is limited by toxicity, which includes both the disruption of gastrointestinal fluid balance and accumulation of toxins in the central nervous system (van Ginkel, F., et al., J Immunol, 2000. 165(9): p. 4778-82).

The well-known potent mucosal adjuvants CT and CpG used for comparisons with compositions of this invention activate immune responses through the cellular toll like receptor 9 (“TLR9”) and the ganglioside receptor (“GM-1”) respectively. The activation and signalling of GM-1 and TLRs is dependant on cell membrane lipid rafts association, which permit the co-localisation of proteins and signalling molecules (Fujinaga, Y., et al., Molecular Biology of the Cell. 2003; Orlandi, P. A. and P. H. Fishman, J. Cell Biol. 1998. p. 905-15; Wolf, A. A., et al., J Biol. Chem. 2002. p. 16249-56; Triantafilou, M., et al., J Cell Sci. 2002. p. 2603-11; Triantafilou, M., et al., J Biol. Chem. 2004. p. 40882-9; Dolganiuc, A., et al., Alcohol Clin Exp Res. 2006. p. 76-85; Latz, E., et al., Nat. Immunol. 2004. p. 190-8).

Lipid rafts are composed of both sphingolipids and cholesterol containing a high proportion of saturated fatty acids, causing a more densely packed area than surrounding unsaturated phospholipids (Simons, K. and W. L. Vaz, Annual review of biophysics and biomolecular structure. 2004. p. 269-95; Dykstra, M., et al., Annu Rev Immunol. 2003. p. 457-81). Free fatty acids intercalate within membrane bilayers directly becoming organised into different domains according to their structure, and the fatty acid content is dynamic due to a high turn over of fatty acids (Klausner, R. D., et al., J Biol. Chem. 1980. p. 1286-95). The incorporation of saturated fatty acids directly influences membrane cellular signalling mechanisms by facilitating the formation of lipid rafts and conversely are inhibited by a high proportion of unsaturated fatty acids (Stulnig, T. M., et al., J Cell Biol, 1998. 143(3): p. 637-44; Stulnig, T. M., et al., J Biol. Chem. 2001. p. 37335-40; Weatherill, A. R., et al., J Immunol, 2005. 174(9): p. 5390-7).

### Liposomes

To protect vaccine viability researchers have explored the development of a number of delivery vehicles including inert particles, liposomes, live vectors and virus like particles (“VLPs”) (Bangham, A. D. and R. W. Home, J Mol. Biol. 1964. p. 660-8; Niikura, M., et al., Virology. 2002. p. 273-80; Guerrero, R. A., et al., J. Virol. 2001. p. 9713-22).

Liposomes and lipid vesicles have also been explored for use with vaccines, particularly with small immunogenic components that may be readily encapsulated. Generally, liposomes and vesicles are not useful for encapsulation of large antigens such as live microorganisms. Moreover, liposomes and vesicles are costly and time consuming to produce, and the extraction procedures used in their preparation may result in alteration of the chemical structure or viability of vaccine preparations and hence their immunogenicity. For example, heat and solvents may alter the biological integrity of immunogenic components such as proteins.

Liposomes are typically small (in the micrometer size range), and are spherical lemellar structures having an inside in which antigens or other materials can be placed. Liposomes are made by mixing lipids with an aqueous solution containing the antigen or other material. After vortexing the mixture, the lipids in the mixture tend to spontaneously form the typical liposomal structure. In some cases, detergents can be added to aid in the mixing of lipid components with aqueous phase components. Upon dialysis to remove detergent, the lipid and aqueous phases tend to separate, with the lipid spontaneously forming the liposomal structure encapsulating the aqueous phase. Liposomes are then typically held in suspension for use.

Immunological responses to liposomal vaccination are highly dependent on the physicochemical properties of the lipids, and therefore a number of sophisticated and complex techniques are employed including reverse-phase evaporation, ether vaporisation, freeze-thaw extrusion and dehydration-rehydration (Szoka, F. and D. Papahadjopoulos, Proc Natl Acad Sci USA. 1978. p. 4194-8; Deamer, D. and A. D. Bangham, Biochim Biophys Acta. 1976. p. 629-34; Chapman, C. J., et al., Chem Phys Lipids. 1991. p. 201-8; Sou, K., et al., Biotechnol Prog. 2003. p. 1547-52; Kirby, C. J. and G. Gregoriadis, Journal of microencapsulation. 1984. p. 33-45).

PCT International Patent Application No: PCT/KR00/00025 (WO 00/41682; herein after “Kim”) discloses a “lipophilic microparticle” (i.e., liposome) incorporating a protein drug or antigen. The microparticles have a size ranging from 0.1 to 200 μm. The lipophilic microparticles may be prepared by coating a solid particle containing an active ingredient with a lipophilic substance in aqueous solution or with use of an organic solvent. Resulting compositions include oil-in-water emulsions suitable for injection. Unfortunately, the microparticles of Kim are not suitable for oral ingestion. They are further not well suited for providing protection of antigens as they pass through the digestive system. As a result, the microparticles of Kim do not provide effective oral immunity of the mucosae.

Additionally, methods for making liposomes require sophisticated manufacturing techniques, which limits the cost effectiveness of large-scale production (Szoka, F. and D. Papahadjopoulos, Proc Natl Acad Sci USA. 1978. p. 4194-8; Deamer, D. and A. D. Bangham, Biochim Biophys Acta. 1976. p. 629-34; Chapman, C. J., et al., Chem Phys Lipids. 1991. p. 201-8; Sou, K., et al., Biotechnol Prog. 2003. p. 1547-52; Kirby, C. J. and G. Gregoriadis, Journal of Microencapsulation. 1984. p. 33-45).

Liposomes have limited use in oral immunization, in part because they are fragile, and because the antigen in the aqueous interior compartment can degrade with time. Additionally, the lipids typically used to make liposomes are those that are liquid at room temperature, and thus, are generally in liquid form under conditions of storage. These features limit the shelf life of liposomal-based vaccines.

### Immune Stimulating Complexes

Immuno-stimulating complexes known as ISCOMS® are composed primarily of phospholipids and cholesterol molecules with defined polar and non-polar regions. Additionally, ISCOMS® contain the highly immunogenic adjuvant saponin (Quil A) (Morein, B., et al., Nature. 1984. p. 457-60). Phospholipids form spherical rings, producing lipid bilayers held together by hydrophobic forces that surround and encapsulated various antigens within an aqueous phase. In both systems it is critical to maintain membrane integrity otherwise antigen is released into the local environment and subject to degradation. Therefore, maintenance of optimal storage conditions is essential for vaccines viability and delivery. ISCOMS® require less complicated manufacturing techniques than liposomes such as dialysis, ultrafiltration and ultra-centrifugation (Sjölander, A., et al., Vaccine. 2001. p. 2661-5). These methods do not necessarily result in the spontaneous incorporation of antigens.

Additionally, another adjuvant has been recently described. ISCOMATRIX® is a lipid adjuvant similar to ISCOMS®. However, ISCOMATRIX® does not physically incorporate antigens but is co-administered as an adjuvant to induce immunity through the immunogenic properties of saponin, so it is not used as a delivery vehicle for the protection of the vaccine antigen(s) during oral immunization (Skene, C. D. and P. Sutton, Methods. 2006. p. 53-9). Liposomes and ISCOMS® have been experimentally used to deliver vaccines via a number of routes, including intramuscular, subcutaneous, intranasal, oral and transcutaneous (Mishra, D., et al., Vaccine. 2006; Wang, D., et al., J Clin Virol. 2004. p. S99-106; Ferrie, Y., et al., Journal of liposome Research. 2002. p. 185-97).

### Lipid Compositions as Oral Vaccine Adjuvants and Carriers for Mucosal Immunization

The difficulties in producing mucosal immunity described above have been unexpectedly overcome by lipid-containing compositions of this invention. Instead of using typical short-chain lipids of the prior art (e.g., oils) or phospholipids of the prior art, we found that the use of long-chain fatty acids as a lipid matrix to hold antigens is suspension have distinct advantages over prior art compositions. First, long-chain fatty acids are more resistant to degradation in the gastrointestinal tract, and thereby provide a protective milieu in which antigens retain their native conformation, thereby increasing an immunogenic response in mucosae.

Humans and other animals consume lipids as part of their daily diet and the digestion of fats (triacylglycerols) is a normal metabolic process. Lipids are barely broken down in the stomach by gastric acids and about 90% of lipid digestion occurs in the intestinal tract by bile salts and lipases (Erickson, R. H. and Y. S. Kim, Annu Rev Med. 1990. p. 133-9).

During oral immunization, saturated fatty acids are not as easily incorporated as unsaturated fatty acids into bile salt micelles, therefore are not readily absorbed by enterocytes. Excess luminal saturated fatty acids may be non-specifically transported with vaccine components across specialized microfold “M” cells. Within the sub epithelial dome the saturated fatty acid portion of a lipid matrix is incorporated in membrane bilayers of antigen presenting cells (APCs), promoting the up regulation of functional GM-1 receptor and TLR complexes. Enhanced protection from lipid formulated vaccines of this invention may be two fold both through the physical delivery of intact antigen and the activation of APCs by mucosal adjuvants.

In contrast with many prior art lipid compositions for pharmaceuticals and vaccines, the lipid compositions of this invention are composed of triglycerides, not phospholipids. Triglycerides do not contain polar and non-polar regions therefore do not organize into concentric spherical bilayers. Instead, lipids used in vaccines of this invention can form a mesh-like matrix in which vaccine components become entrapped. This provides the physical protection of lipid-incorporated antigens during exposure to varying storage factors, such as humidity and moisture, and during the harsh acidic environment of the stomach.

Lipids employed in the formulations above are desirably suitable for animal or human consumption and may be selected from a broad range of natural (vegetable or animal derived), or synthetic lipid products including oils, fats and waxes. In developing new vaccines, avoiding the generation of adverse side effects is a main determinant for trials of vaccine use in humans. The use of ‘safe’ subunit antigens without the co-administration of toxic adjuvants would be ideal. Lipid formulations of this invention are manufactured using food or pharmaceutical grade dietary fatty acids that are not associated with any adverse side effects. Oral immunization with such lipid-formulated MOMP induced significant protection of the respiratory and genital mucosa from Chlamydia infection. Additionally, the incorporation of killed whole-cell H pylori into lipid formulations of this invention elicited protection at the gastrointestinal tract following live bacterial challenge with H. pylori SS1 (FIGS. 3 and 5). This degree of protection observed with lipid C and H pylori antigen was about 25% reduction in bacteria recovered, which represents a decreased bacterial load in the animal. Immunization resulted in a significant reduction in bacteria recovered from stomach tissue six weeks following intra-gastric inoculation with Helicobacter pylori SS1.

Killed whole-cell organisms are composed of numerous antigens that are not identified, nor isolated for their immunogenicity. Purified MOMP however, is an immunodominant surface antigen containing both class I and class II T cell epitopes (Caldwell, H. D., et. al., Infect Immun. 1981. p. 1161-76). Lipid formulation of MOMP according to this invention elicited immune responses that partially protected mice against both respiratory and genital chlamydial infections.

The manufacture of vaccines according to this invention is a simple inexpensive mechanical process with no specialised expertise or equipment requirements. Although liposomes and ICOMS® are highlighted as a cheaper options compared to other non-lipid delivery vehicles, the simplicity of the compositions of this invention can provide an even more inexpensive alternative.

In some embodiments, a lipid formulation can be liquid at temperatures above about 30° C. That is, the lipid can be selected to achieve melting point at physiological temperature in the animal to which it is administered, most usually by the oral route. Desirably, the lipid will be in the form of a solid at 10° C.-30° C. at atmospheric pressure, and preferably is still solid at from 20° C. to 30° C. at atmospheric pressure. However the melting temperature of lipid is not exclusive and may include oils, fats and waxes with a range of melting temperatures.

In some embodiments, lipids for use herein can undergo a transition from the solid phase to a liquid phase between about 30° C. and human physiological temperature of about 37° C. Summaries of lipid phase behaviour are available in the art. Accordingly, a skilled reader can select a lipid having the desired properties and melt point based on information in the art and simple experiment.

In general, suitable lipid formulations can include triglycerides such as glyceryl esters of carboxylic acids, compounds consisting of an aliphatic chain and a —COOH end, and saturated and non-saturated fatty acids and mixtures thereof.

In some embodiments, triglycerides can contain primarily C₈ to C₂₀ acyl groups, for example myristic, palmitic, stearic, oleic, linoleic, parinic, lauric, linolenic, arachidonic, and eicosapentaenoic acids, or mixtures thereof.

In some embodiments, lipid formulations useful in the invention include longer chain fatty acids, for example, C₁₆-C₁₈. Long chain fatty acids have been found to be more effective in protecting organisms such as BCG in vaccines given to mice and possums. Viewed in this way, lipid formulations preferred for use in the invention contain: about 30% to about 100%, alternatively about 60% to about 100%, alternatively about 80% to about 100%, and in other embodiments, about 90% to about 100% C₁₆ and/or C₁₈ fatty acids.

In other embodiments, C₁₆ fatty acids can represent from about 10% to about 40%, alternatively about 20% to about 35%, and in other embodiments from about 25% to about 32% of the total fatty acid content. C₁₈ fatty acids can represent from about 30% to about 90%, alternatively from about 50% to about 80%, and in still other embodiments, from about 60% to about 70% C₁₈ of the total fatty acid content.

Still other embodiments have lipid formulations containing less than about 35% C₁₄ fatty acids or shorter, alternatively less than about 25%, and in yet further embodiments, less than about 10%.

The chain length of lipids in certain embodiments are less than about 5% fatty acids with C₁₄ chains or shorter, about 25% to about 32% C₁₆ fatty acids, and from about 60% to about 70% C₁₈ fatty acid chains.

In certain embodiments, lipid formulations for use in the invention may contain: saturated fatty acids in an amount from about 20% to about 60%, alternatively from about 30% to about 55%, and in still other embodiments, from about 40% to about 50%. Monounsaturated fatty acids can be in the range of about 25% to about 60%, alternatively from about 30% to about 60%, and in yet other embodiments, from about 40% to about 55%. Polyunsaturated fatty acids can be in the range of about 0.5% to about 15%, alternatively from about 3% to about 11%, and in further embodiments, in the range of about 5% to about 9%.

Some embodiment of the invention include about 40% to about 50% saturated fatty acids, about 40% to about 50% monounsaturated fatty acid, and about 5% to about 9% polyunsaturated fatty acid.

In some embodiments, a lipid formulation for use in the invention has about 3% myristic acid, about 26% palmitic acid, about 15% stearic acid, about 40% oleic acid, and about 6% linoleic acid as determined by HPLC analysis.

In further embodiments, a lipid formulation of the invention has about 1% myristic acid, about 25% palmitic acid, about 15% stearic acid, about 50% oleic acid an about 6% linoleic acid

(“Lipid C”). In some of these embodiments, compositions contain Lipid C and MOMP. In other embodiments, compositions contain Lipid C and H pylori antigens.

Other embodiments of this invention comprise a variation of Lipid C, “Lipid Ca,” having 2.8% myristic acid, 22.7% palmitic acid, 2.5% palmitoleic acid, 1.1% daturic acid, 15.9% stearic acid, 38.0% oleic acid (C18:ln-7), 1.7% oleic acid (C18:ln-9) and 4.0% linoleic acid, having a total saturated fat composition of 42.4%, a monounsaturated fat composition of 42.2%, and a polyunsaturated fat composition of 4.0%. In some of these embodiments, compositions contain Lipid Ca and MOMP. In other embodiments, compositions contain Lipid Ca and H pylori antigens.

Alternatively, a lipid formulation of this invention includes hydrogenated coconut oil (“Lipid K”). Some Lipid K-containing compositions include 7.6% caprylic acid (C8:0), 6.8% capric acid (C10:0), 45.1% lauric acid (C12:0), 18.3% myristic acid (C14:0), 9.7% palmitic acid (C16:0), 2.7% stearic acid (C18:0), 7.7% oleic acid (C18:1), and 2.3% linoleic acid (C18:2) and having a melting point of about 27.1° C. In some of these embodiments, compositions contain Lipid K and MOMP. In other embodiments, compositions contain Lipid K and H pylori antigens.

Other variants of Lipid K-containing embodiments comprise fatty acids having a composition by weight of 6.5% caproic acid (C6:0), 5.4% capric acid (C10:0), 44.5% laurate (C12:0), 17.8% myristic acid (C14:0), 9.8% palmitic acid (C16:0), 11.5% stearic acid (C18:0), 2.2% oleic acid (C18:0) and a total saturated fat composition of 95.5%, and a monounsaturated fat composition of 2.2%. In some of these variant Lipid K embodiments, compositions contain Lipid K and MOMP. In other of these variant Lipid K embodiments, compositions contain Lipid K and H. pylori antigens.

In still other alternatives, a lipid formulation of this invention includes pharmaceutical grade hydrogenated coconut oil (“Lipid PK”). In some Lipid PK-containing embodiments comprise fatty acids having a composition by weight of 7.0% caproic acid, 5.8% capric acid, 45.0% laurate, 18.2% myristic acid, 9.9% palmitic acid, 2.9% stearic acid, 7.6% oleic acid and 2.3% linoleic acid and a total saturated fat composition of 88.8%, a monounsaturated composition of 7.6%, and a polyunsaturated fat composition of 2.3%. In some of these embodiments, compositions contain Lipid PK and MOMP. In other embodiments, compositions contain Lipid PK and H pylori antigens.

In additional alternatives, a lipid formulation of this invention includes “Lipid SPK” having a composition by weight of 6.7% caproic acid, 5.6% capric acid, 44.3% laurate, 17.9% myristic acid, 9.6% palmitic acid, 3.0% stearic acid, 8.4% oleic acid and 2.6% linoleic acid, and a total saturated fat composition of 87.3%, a monounsaturated fat composition of 8.4%, and a polyunsaturated fat composition of 2.6%. In some of these embodiments, compositions contain Lipid SPK and MOMP. In other embodiments, compositions contain Lipid SPK and H pylori antigens.

Compositions are easily manufactured from readily available lipid components. In certain embodiments, lipid compositions of this invention consist of both purified and fractionated triglycerides that when warmed to a molten state above 37° C. allows for the incorporation of various antigens and immunomodulators, however once cooled it forms a solid stable phase (Aldwell, F. E., et al., Infect Immun, 2003. 71(1): p. 101-8). The manufacture of vaccines of this invention is a simple inexpensive mechanical process with no specialised expertise or equipment requirements. Although liposomes and ICOMS® are highlighted as a cheaper options compared to other non-lipid delivery vehicles, the simplicity of manufacturing vaccines of this invention provides an even more inexpensive alternative.

Lipid formulations of this invention are useful in the preparation of immunogenic compositions, and in protecting antigens within the composition from degradation. The lipid formulation is especially useful in maintaining viability of live organisms, particularly bacteria. The lipid formulation acts to maintain the organisms in a live, but dormant state. This is particularly important for vaccines comprising live organisms formulated for oral administration. The lipids also maintain antigens in a uniform suspension. That is, in the compositions of the invention the immunogenic components can be uniformly distributed throughout a solid or paste like lipid matrix. The lipids also protect the antigens from destruction by gastrointestinal secretions when orally administered. Protection from macrophage attack is also likely when administered by other routes such as subcutaneously. This allows for uptake of the antigens and particularly live organisms through the gastrointestinal mucosa, and subsequent replication of organisms in the host.

The compositions of this invention are more resistant to degradation during storage conditions. For example, liposomes are know to aggregate upon lengthy storage, and in some cases, liposomal preparations may require that either positive or negative charges must be built into the lipisome, to provide electrostatic repulsion that favors maintaining the liposomes in suspension.

### Immunogenic Components

Generally, a vaccine includes one or more substances to which an immune response can be generated. Such substances include lipids, proteins, carbohydrates or other organism-specific components. The requirements are simple; the substance must be capable of being presented to an immune cell and the immune cell must be capable of producing an immune response. In many cases, a protein is the immunogen.

In other cases, living organisms are used. Effective protection following subcutaneous immunization of humans via this route is often highly variable for organisms such as BCG which ranges from 0-80% (Colditz, G. A., et al., Pediatrics. 1995. p. 29-35; Colditz, G. A., et al., JAMA. 1994. p. 698-702; Fine, P. E., Lancet. 1995. p. 1339-45).

Recently one of the inventors and colleagues used a lipid-based oral delivery system, Lipid C, for oral vaccine delivery in animals (Aldwell F E, et al., Infect Immun 2003; 71(1):101-8; Aldwell, F., et al., Vaccine, 2003. 22(1): p. 70-6; PCT International Patent Application No: PCT/NZ2002/00132) U.S. Pat. No. 7,758,869. Feeding of live Mycobacterium bovis Bacille Calmette-Geurin (BCG) vaccine incorporated in Lipid C to mice produced resistance to infection (Aldwell, F E, et al., Infect Immun 2003; 71(1):101-8). Similar results were found in white tail deer (Nol P, et al. J Wildl Dis 2008 April; 44(2):247-59), guinea pigs (Clark S, et al., Infect Immun 2008 Jun. 2) and brushtail possums (Aldwell F E, et al., Vaccine 2003 Dec. 8; 22(1):70-6) where immunization with lipid-C based vaccines protected against aerosol challenge with live M bovis. Levels of protection were observed to be greater than those seen in animals immunized with non-incorporated BCG, and were equivalent to BCG administered by the subcutaneous route and was associated with strong interferon gamma (IFNγ) production by systemic and mucosal T cells. Because Lipid C-incorporation of BCG, a live attenuated vaccine organism, greatly increased its immunogenicity following oral delivery, we determined that Lipid C can also enhance the immune response to defined subunit protein antigens of Chlamydia or Helicobacter delivered by the oral route. However, in the case of pathogenic organisms, it is important to ensure that the immunized animal does not become seriously and adversely affected by the pathogen.

In contrast with the successful immunization against BCG using live organisms described above, use of non-living, non-replicating BCG antigens did not provoke an immunogenically effective immune response (M. I. Cross, et al., Immunology and Cell Biology, 1-4, 13 Nov. 2007). Thus, there is an additional problem in the art, namely the production of effective immune responses to pathogenic organisms by the use of non-infective antigens.

A number of vaccines rely on the use of freeze-dried preparations of organisms. For example, a current vaccine for human TB is based on freeze-dried preparations of a live attenuated bacterium called Bacille Calmette Guerin (“BCG”). However, it has been shown that freeze-drying procedures result in 30% to 50% loss of viability of BCG and impaired recovery of remaining live bacteria (Gheorghiu, M., et al., Dev. Biol. Stand. Basel, Karger. 87:251-261). A composition which retains greater viability of organisms prior to use would contribute greatly to the effectiveness of such vaccines.

In other cases, it is desirable to use specific proteins from an organism. In the case of Chlamydia, the major outer membrane protein (MOMP) is used as a immunogenic compound, because this protein is implicated in the function of the Chlamydial organism.

### Chlamydial Antigenic Components

The outer chlamydial cell wall contains several immunogenic proteins, including a 40-kilodalton (kDa) major outer membrane protein (MOMP), two cysteine rich proteins the 60- to 62-kDa Outer Membrane Complex B Protein (OmcB) and the 12- to 15-kDa Outer Membrane Complex A Protein (OmcA) a 74 kDa species-specific protein, and 31- and 18-kDa eukaryotic cell-binding proteins, which share the same primary sequence.

Hyperimmune mouse antiserum against the 40-kDa MOMP protein from serotype L2 react with elementary bodies of C trachomatis serotypes Ba, E, D, K, L1, L2, and L3 during indirect immunofluorescence but failed to react with serotypes A, B, C, F, G, H, I, and J or with C psittaci. Indeed, cloning and sequencing of the C trachomatis MOMP gene revealed the same number of amino acids for serovars L2 and B, while the MOMP gene of serovar C contained codons for three additional amino acids. The diversity of the chlamydial MOMP was reflected in four sequence-variable domains, two of which are candidates for the putative type-specific antigenic determinants. The basis for MOMP differences among C trachomatis serovars were clustered nucleotide substitutions for closely related serovars and insertions and deletions for distantly related serovars. When MOMP is inserted into the outer elementary body envelope, exposed domains of MOMP serve as both serotyping and protective antigenic determinants. Predominantly conserved regions of C and B serotypes are interspersed with short variable domains.

Serovars D, E, F, G, H, I, J, and K are known to be associated with human disease. Vaccination against serovars E, F, and G together would protect approximately 75-80% of individuals. Serovars D, E, F, G, H, I, J, K and L are associated with genital infection by Chlamydia, and serovars A, B, C, D, E, F, G, H, I, J, and K are associated with ocular infections. Chlamydia pneumonae is also associated with Alzheimer's disease, coronary artery disease and asthma.

Three monoclonal antibodies that recognize epitopes on cysteine-rich membrane proteins interact with all 15 human C trachomatis serotypes, establishing the species specificity of this antigen. Monoclonal antibodies to OmcA showed biovar specificity and species specificity. The OmcB and OmcA cysteine-rich proteins are highly immunogenic in the natural infection, but the antibodies do not neutralize the infectivity of C trachomatis elementary bodies.

Thus, a candidate antigen for the development of a vaccine is chlamydial major outer membrane proteins (“MOMP”). We unexpectedly found that animals orally immunized with chlamydial MOMP either incorporated in Lipid C or admixed with the strong mucosal adjuvants CT and CpG oligodeoxynucleotides were protected against intravaginal challenge with Chlamydia muridarum. Surprisingly, we found that the combination of Lipid C and CpG/CT and MOMP improved the immune responses to challenge with C. muridarum greater than observed for either Lipid C plus MOMP or CpG/CT plus MOMP.

In addition to MOMP directed immunization, Chlamydia infections can be reduced by immunizing susceptible animals using immunogenic components other than those of MOMP. Several distinct immunogenic components have been recognized in C trachomatis and C psittaci, some group specific and others species specific. Detergents have been used to extract antigens from elementary bodies and reticulate bodies. Chlamydia pneumoniae (TWAR organism) is serologically unique and differs from C trachomatis species and all C psittaci strains.

It can be appreciated that immunogenic components from C. muridarum can also be incorporated into compositions useful for testing in a murine model of infection by Chlamydia. It can also be appreciated that there are numerous combinations of Chlamydia antigens that can be mixed together and incorporated into an orally active vaccine of this invention.

### Helicobacter Antigen Components

To provide a broad-spectrum innoculum, ultraviolet (“UV”)-killed whole-cell Helicobacter pylori Sydney Strain 1 (H pylori SS1) can be used. Using the whole organism avoids the necessity of determining which of the Helicobacter antigens are immunogenic. It can be appreciated that other strains of Helicobacter can be used without departing from the scope of this invention.

### Embodiments of this Invention

Manufacture of Lipid Compositions of the Invention

A composition of this invention may be prepared using techniques known in the art. Conveniently, the lipid formulation is heated to liquefy if required, and the immunogenic component(s) and other ingredients (when used) as described above are added. Dispersal of the immunogenic composition may be achieved by mixing, shaking or other techniques that do not adversely affect the viability of the immunogenic component. In some embodiments, the antigen is uniformly dispersed throughout the lipid formulation.

Alternative compositions for use in the invention can be essentially free of aqueous components including water. The term “essentially free” as used herein means that the composition contains less than about 10% aqueous components, and preferably less than about 5% aqueous components. As indicated above, the presence of components, particularly aqueous solvents, reduces the protective effect of the lipid formulation especially in the gut.

An immunogenic composition of the invention can also be useful for generating a response to a second or further immunogenic molecule of a type as indicated above for the immunogenic component, particularly those that are weakly immunogenic. This may be achieved by co-delivery of the second or further immunogenic molecule in an immunogenic composition by conjugating the immunogenic molecule to another immunogenic component of the composition. Conjugation may be achieved using standard art techniques. In particular, an antigen of interest may be conjugated to an immunogenic carrier or adjuvant by a linker group which does not interfere with antibody production in vivo. The immunogenic carrier or adjuvant may be any of the immunogenic components including the organisms identified above but are preferably Mycobacterium, and more preferably BCG. Suitable linker groups include mannose receptor binding proteins such as ovalbumin and those that bind to Fc receptors. The second or further immunogenic molecule is preferably a protein or peptide. A particularly preferred protein is an immunocontraceptive protein. The lipid again acts as the delivery matrix. When the composition is administered an enhanced immune response to the conjugated molecule or co-delivered molecule results.

The term “animal” as used herein refers to a warm-blooded animal, and particularly mammals. Humans, dogs, cats, birds, cattle, sheep, deer, goats, rats, mice, rabbits, possums, badgers, guinea pigs, ferrets, pigs and buffalo are examples of animals within the scope of the meaning of the term. Monogastric and ruminant animals in particular are contemplated within this term.

The term “antigen” as used herein in the context of vaccine compositions of this invention is equivalent to the term “immunogen,” and refers to a substance capable of eliciting an immune response in an animal or a substance that can be specifically bound by an antibody or immune system cell of an animal that had been immunized against the substance.

Formulations for a wide range of delivery routes may also include, in addition to a lipid formulation and one or more immunogenic components, additives such as fillers, extenders, binders, wetting agents, emulsifiers, buffing agents, surfactants, suspension agents, preservatives, colourants, salts, antioxidants including mono sodium glutamate (MSG), vitamins such as vitamin E, butylated hydroxanisole (BHA), albumin dextrose-catalase (ADC), protective coatings, attractants and odourants, and agents to aid survival of organisms or other antigens contained in the lipid but are not limited thereto.

Protective coatings or enterocoatings may be selected, for example, from gels, paraffins, and plastics including gelatin. The coatings further aid in the prevention of exposure to gastric acids and enzymes when the oral administration route is selected.

When used for oral administration, the formulation may also include additives which, for example, improve palatability, such as flavouring agents (including anise oil, chocolate and peppermint), and sweeteners (including glucose, fructose, or any other sugar or artificial sweetener). It can be appreciated from the foregoing that the immunogenic component may be a complex of proteins or peptides, or the like.

In one embodiment, the composition includes at least two immunogenic components selected from any of those identified above, and may include multiple combinations of subunit antigens. Three or more immunogenic components are feasible.

The concentration of the immunogenic component(s) in the composition may vary according to known art protocols provided it is present in an amount which is effective to stimulate an immune response on administration to an animal. In particular, an immune response in the gut associated lymphoid tissue of the small intestine. In the case of Mycobacteria a range of from 1×10⁵ to 1×¹⁰ colony forming units (CFU)/ml is appropriate. Preferably, the concentration is from 1×10⁷ to 1×10⁹ CFU/ml. For protein and peptide type antigens, including Chlamydia MOMP and H. pylori antigens, a range of from 10-1000 μg per gram of formulation is appropriate. For virus-type antigens a range of 1×10³ to 1×10¹⁰, preferably 1×10⁵ to 1×10⁸ Plaque Forming Units (PFU)/ml is appropriate. The immune response may be humoral (e.g., via soluble components such as antibodies or immune mediators) or cell mediated including a mucosal immune response.

For example, in a series of in vivo experiments, we studied BALB/c mouse models of gastric Helicobacter and Chlamydia genital, gastrointestinal, and respiratory infections. These systems are well known to be related to human disorders, and were used to determine the effectiveness of compositions and methods of this invention for induction of protective mucosal immunity.

Killed or subunit vaccines provide a safe alternative to live-attenuated vaccines, with less associated adverse reactions. Oral immunization with either killed whole-cell H. pylori or purified Chlamydial MOMP alone does not protect mucosal surfaces following live bacterial challenge. We therefore investigated the potential of lipid formulated compositions to induce mucosal immunity following oral immunization with non-living whole cell antigens and defined protein antigens, with and without addition of the potent mucosal adjuvants CT and CpG-ODN for protection against Helicobacter and Chlamydia mucosal infections.

In this disclosure, we demonstrated that oral immunization of mice using vaccines formulated in lipid compositions of this invention enhanced protection against live bacterial challenge at multiple mucosal surfaces. Lipid-formulated killed whole-cell or protein antigens admixed with CpG-ODN and cholera toxin (CpG/CT) elicited protection not just locally in the gut, but also at the anatomically distant genital and respiratory surfaces.

The main limitation for oral immunization is the determination and maintenance of optimal antigen doses for vaccination. Orally administered antigens become absorbed from the intestinal lumen at an unpredictable and somewhat low rate. Protein antigens introduced to the GALT generally results in the induction of tolerance rather then immunity (Challacombe, S. J. and T. B. Tomasi, J Exp Med. 1980. p. 1459-72). For these reasons, oral vaccines required large doses of antigen and/or adjuvants to boost adaptive immunity. Unfortunately, a high antigen dose and the presence of toxic adjuvants increase the likelihood of adverse side effects. In this study, both chlamydial MOMP and killed whole-cell H pylori administered alone were unable protect mucosal surfaces from bacterial infection and results are comparable to non-immunized controls. The co-administration of potent mucosal adjuvants is desirable for the induction of protective immunity using the oral route.

Because CT is toxic to human beings, it is an undesirable component of a human immunogenic composition of this invention. However, because CT is not toxic to certain other animals, CT can be included in immunogenic compositions for induction of immunity in other animals.

Because CpG is an oligonucleotide, its toxicity is less than that of CT, and CpG can be incorporated into immunogenic compositions of this invention for human use. Thus, for induction of immunity in human beings, compositions of this invention can include a chlamydial antigen, a lipid formulation and CpG oligonucleotide. In other embodiments, a composition of this invention can include an antigen from either Chlamydia or H. pylori, a lipid formulation comprising Lipid C, Lipid Ca, Lipid K, Lipid Pk or Lipid SPK and CpG oligonucleotide.

### Utility

The compositions and methods of this invention are useful for providing immunity against organisms that infect mucosae. Because lipid-containing compositions of this invention provide mucosal immunity, they are well suited for multiples uses. Immunogenic compositions of this invention include a lipid formulation that maintains antigens in a stable matrix, through which they can be uniformly dispersed. This facilitates administration of consistent doses of antigen, avoiding dose dumping and ineffective low dosing. The lipid formulation also protects the antigens from degradation by stomach acids and digestive enzymes. Losses in viability of immunogenic components in lipid based formulations are also significantly lower than those reported for freeze-dried products. Storage under humid or moist conditions can also be achieved without deterioration because of the hydrophobic properties of the formulation.

Stability of immunogens in vaccine preparations is important for inducing strong and long lasting protective immunity. This may be achieved using the compositions of the invention. The compositions are also simple to prepare, more affordable to produce, and find increased consumer acceptance and safety where the use of needles and syringes can be avoided.

This disclosure provides direct evidence of induction of mucosal immunity against Chlamydia and Helicobacter infection. We unexpectedly found that oral immunization of mice with MOMP incorporated in Lipid C was as effective as immunization with MOMP mixed with the potent mucosal adjuvants CT and CpG at protecting BALB/c mice against a genital challenge with C. muridarum. This was surprising given the lower levels of IFNγ production and genital tract antibody levels observed in mice immunized with Lipid C compared to those immunized with CT/CpG plus MOMP. Importantly, lipid C incorporation of both MOMP and CT/CpG resulted in even greater levels of protection suggesting a synergistic effect when adjuvants were combined with lipid C.

We also unexpectedly found that oral immunization with lipid compositions of this invention incorporating ultraviolet (“UV”)-killed whole-cell Helicobacter pylori Sydney Strain I (H. pylori SS1) were effective in inducing mucosal immunity against infection with live organisms. Further, the above compositions admixed with cholera toxin (“CT”) and bacterial CpG oligonucleotides (“CpG-ODN” or “CpG”) also induced protective immunity at the gastrointestinal, respiratory and genital mucosae.

The degree of protection afforded by lipid/antigen compositions of this invention were comparable to those observed with the antigen plus CpG/CT as the antigen. Thus, the lipid compositions of this invention provide mucosal immunity without the harmful side effects of CT and CpG.

Mucosal protection was associated with strong splenic IFNγ cytokine expression and antigen specific antibody in both serum and mucosal secretions. Predominately IgG was detected in serum and BAL fluid, whilst IgA production was evident in genital lavage and faecal washes. In this study, protection without the addition of the additional adjuvants CT and CpG was also observed following oral immunization using lipid compositions of this invention formulated MOMP. This resulted in a 50% reduction in chlamydial load at both the respiratory and genital tracts following live bacterial challenge. Oral immunization with compositions of this invention effectively elicited protective immunity at multiple mucosal surfaces.

While oral immunization has usually been used to induce protection against gastric, and to a lesser extent, respiratory pathogens, a number of studies have demonstrated that oral immunization can target the female genital tract. Challacombe et al. Vaccine 1997 February; 15(2):169-75) showed that oral immunization with ovalbumin in poly D,L-lactide-co-glycolide (PLG) microparticles elicited significant OVA-specific antibody in vaginal lavage. Oral immunization with live influenza virus elicited virus-specific IgA in homogenates of urinary bladder, uterus, vagina and in uterine washings (Briese V, et al., Arch Gynecol 1987; 240(3):153-7).

Oral immunization of mice with recombinant Salmonella expressing the sperm receptor ZP3 induced ZP-3-specific IgA in vaginal secretions as well as infertility (Zhang X et al., (published erratum appears in Biol Reprod 1997 April, 56(4):1069). Biology of Reproduction 1997; 56(1):33-41).

Oral immunization of mice with PLG micropartices containing an anti-idiotypic antibody against the chlamydial glycolipid exoantigen also partially protected mice against genital challenge with a human strain of C. trachomatis (Whittum-Hudson J A, et al., Nature Medicine 1996; 2(10):1116-21).

All of the above studies required the use of either live (influenza virus) or attenuated (Salmonella) organisms or incorporation of antigen into PLO microparticles to elicit immunity in the reproductive tract. Production of PLG microparticles is expensive and the solvent extraction methods use can destroy the immunogenicity of some protein antigens. Furthermore, the use of live Chlamydia vaccines are unlikely to be approved due to the enhanced inflammatory responses that were seen following trials of live and killed vaccines to prevent trachoma (Grayston J T, Wang S P. Sexually Transmitted Diseases 1978; 5(2):73-7).

Because Lipid C is composed solely of food-grade lipids that are regularly consumed as part of a normal diet and can be easily prepared by simple mixing of components they may offer a significant advantage over other particulate delivery systems such as liposomes and PLO microparticles in terms of ease of preparation and cost. Furthermore, because Lipid C forms a solid below 33° C. it may protect component antigens against degradation during storage thereby prolonging the effective shelf life of the vaccine. Lipid C incorporation of BCG certainly enhanced its viability during prolonged storage at both 4° C. and at room temperature (Aldwell F E, et al., Vaccine 2006 Mar. 15; 24(12):2071-8).

The mechanisms of action of lipid formulations of this invention are not completely understood. Without being bound by any particular mechanism of action however, adjuvant or carrier effects of Lipid C may be due to a number of factors. First, because fats are broken down in the intestine by pancreatic enzymes and not in the stomach (Armand M. Curr Opin Clin Nutr Metab Care 2007 March; 10(2):156-64), compositions of this invention may protect antigens against destruction from gastric digestive processes and acidic pH and deliver antigens to inductive sites such as Peyer's patches in the small intestine or intestinal dendritic cells (Rescigno M, et al. Nat Immunol 2001; 2(4):361-7).

Lipids may also directly affect the functioning of immune cells in these inductive sites through their affects on lipid rafts and on membrane fluidity. Lipid rafts are essential for signalling between immune cells as signalling molecules such as MHC molecules, T cell and B cell receptors need to cluster in lipid rafts for effective cell-cell signalling (Horejsi V. Immunol Rev 2003 February; 191:148-64; Dykstra M, et al., Annu Rev Immunol 2003; 21:457-81; Anderson H A, et al., Nat Immunol 2000 August; 1(2):156-62).

Lipid rafts normally contain higher amounts of saturated fatty acids than surrounding areas of cell membrane and it is possible that lipid raft function may be enhanced by the saturated fatty acids in Lipid C. Conversely, increased proportions of unsaturated fatty acids can increase membrane fluidity, which can enhance phagocytosis thereby potentially increasing antigen uptake by APC (Mahoney E M, et al., Proc Natl Acad Sci USA 1977 November; 74(11):4895-9; Weatherill A R, et al., J Immunol 2005 May 1; 174(9):5390-7; Schweitzer S C, et al. J Lipid Res 2006 November; 47(11):2525-37).

Various lipids have also been reported to have anti-inflammatory effects and to inhibit lymphocyte stimulation (Calder P C, et al., Biochem Soc Trans 1989 December; 17(6):1042-3; Calder P C, et al., Biochem Soc Trans 1990 October; 18(5):904-5). It may be useful to determine which mechanisms are important for the adjuvant effects of Lipid C. The low toxicity of an adjuvant formulated from food grade lipids may provide another significant advantage for mucosal vaccination. Mucosal responses are generally short-lived, due to the short half-life of plasma cells and to the remodelling of tissues in the female reproductive tract as part of the normal reproductive cycle. As such frequent boosting may be required to maintain protective levels of immunity. In these and other studies with Lipid C we observed no adverse reactions to multiple oral doses of Lipid C suggesting that frequent use would be well tolerated.

### Vaccination of Vector Animals

One way of decreasing disease in an animal is to decrease exposure of the animal to the pathogen. In the case of certain pathogens, vector animals can provide a “pool” of pathogens, even if the animal does not show signs or symptoms of the disease. Therefore, vaccination of wildlife, such as possums, badgers, cattle, rodents, deer and the like can be effective in reducing incidence of disease caused by pathogens. To vaccinate vector animals, it can be desirable to deliver the vaccine by the mucosal route. Oral vaccines therefore represent a practical and cost effective delivery option. Thus, in certain embodiments, lipid compositions of this invention can include attractants, flavouring agents and odorants that can be selected based on the vector animal to be vaccinated. Oral vaccination of humans is also a more cost effective method of vaccination and likely to find favour with users.

Additionally, when administered in other ways such as subcutaneously, the lipid formulations of this invention can provide protection from'attack, for example, by macrophages or other scavenger cells. With subcutaneous administration, or administration by injection, the formulation of a lipid depot also allows sustained release to mimic the infection process, and facilitate the mounting of an immune response.

The compositions can be effective in inducing immune responses to a wide range of infectious organisms, including reproductive, optic, gastrointestinal and respiratory pathogens. By way of example, Lipid C can be effective in eliciting immune responses in the genital tract and gastrointestinal tract, and lipid PK can be effective in eliciting immune responses in the upper and lower alimentary tract and the respiratory tract.

The compositions of the invention may also be used as a vaccine delivery system for a wide range of antigens, or for the co-delivery or conjugated delivery of immunogenic molecules, particularly those which for reasons of dose or immunogenicity are poorly immunogenic. The compositions of the invention are also useful as vaccine adjuvants and can be delivered along with conventional adjuvants (e.g., Freund's complete or Freund's incomplete adjuvants).

### EXAMPLES

The following examples are presented to illustrate specific embodiments of this invention. It can be appreciated that persons of ordinary skill in the art can readily adapt the disclosures and teachings herein to produce other embodiments without undue experimentation. All of such embodiments are considered to be part of this invention.

### Example 1

### Preparation of Recombinant Chlamydial MOMP

Chlamydia and its infective properties in mice are very similar to those found in human disease, and therefore, studies of such infections, their properties and their treatments are highly predictive of human therapy. For the chlamydial infection studies, the major outer membrane protein (MOMP) was purified by an adapted method from Berry et al. [Berry, L. J., et al., Transcutaneous immunization with combined cholera toxin and CpG adjuvant protects against Chlamydia muridarum genital tract infection. Infect Immun, 2004. 72(2): p. 1019-28]. Briefly, transformed Escherichia coli (DH5α{pMMM3}) expressing the pMAL-c2 vector encoding recombinant maltose binding protein (MBP)-MOMP fusion protein (generous gift from Harlam Caldwell—Rocky Mountain Labs, Hamilton, Mont. (Su, H., et al., Proc Natl Acad Sci USA, 1996. 93(20): p. 11143-8) is isolated following growth on ampicillin nutrient agar and harvested through sonication (as per Berry et al).

The major outer membrane protein (MOMP) of C. muridarum was purified by the method of Berry et al from transformed E. coli (DH5 cc pMMM3 expressing the pMAL-c2 vector encoding recombinant maltose binding protein (MBP)-MOMP fusion protein (generous gift from Harlan Caldwell, Rocky Mountain Labs, Hamilton, Mont.). C. muridarum is an agent that causes infections of the reproductive tracts of mice.

MOMP was purified and refolded from 8M Urea to PBS (pH 7.2) using dialysis tubing prepared as per manufacturers instructions (Sigma-Aldrich, Castle Hill, Australia). Protein concentration was estimated using Pierce BSA protein estimation kit, and stored at −20° C. until required.

### Example 2

### Antigen Preparation for Immunization Against H. pylori

For the H. pylori infection studies, whole killed antigen was produced using H pylori Sydney Strain 1 (kindly donated by Dr Hazel Mitchell, University of New South Wales (Lee, A., et al., Gastroenterology, 1997. 112(4): p. 1386-97) grown on grown on campylobacter selective agar (CSA) consisting of with 5% (v/v) sterile horse blood in blood agar base No. 2 (Oxoid Ltd., Basingstoke, England) and Skirrow's supplement as published by Sutton et al. (Sutton, P., et al., Vaccine, 2000. 18(24): p. 2677-85). Plates were harvested into sterile PBS and concentration (colony forming units per ml) was established using McFarland's standards. Live bacteria were inactivated through exposure to ultraviolet (UV) radiation and lack of bacterial viability established through culture. Killed whole cell H pylori was stored at −20° C. until use.

### Example 3

### Preparation of Immunization Compositions I

A lipid C formulation consisting of fractionated and purified tri-glycerides containing of 1% myristc acid, 25% palmitic acid, 15% stearic acid, 50% oleic acid and 6% linoleic acid was supplied by Immune Solutions Ltd (Dunedin, New Zealand). For chlamydial infection studies 200 μg MOMP was used as an antigen and 10⁷ cfu of killed whole-cell H. pylori was used for Helicobacter immunizations. Immunization groups included: (1) Non-immunized control animals, (2) animals treated with antigen admixed with 10 μg CpG-ODN 1826 (5′-TCC ATG ACG TTC CTG ACG TT-3′; SEQ ID NO:1) (Geneworks) and 10 μg cholera toxin (Sapphire Biosciences) (CpG/CT), (3) lipid C and antigen alone, and (4) lipid C plus antigen admixed with CpG/CT. Lipid C, MOMP, CT and CpG were mixed using a 3-way stopcock and 2 syringes such that 150 ul of Lipid C contained 200 ug MOMP either alone or together with CT (10 ug) and CpG (10 ug). For the non-lipid formulated vaccines MOMP, either alone or combined with CT and CpG were prepared in PBS. All formulated vaccines were prepared before first immunization and stored until required at 4° C. Unformulated vaccines were required to be prepared on the day of each immunization.

### Example 4

### Immunization of Mice

Specific pathogen free (SPF) female BALB/c mice were obtained from the Animal Resource Centre (ARC) (Perth, Wash.). Animals were housed under standard day-night cycle and provided with sterile food and water ad libitum. The University of Newcastle's Animal Care and Ethics Committee approved all procedures.

Mice were immunized with 150 μl immunization solutions 3 times at weekly intervals by oral gavage using a ball-ended needle under isofluorane anaesthesia, and boosted 3 weeks later. Control animals were treated identically, but were not immunized.

Mice infected with Chlamydia or Helicobacter represent art-recognized systems that are reasonably predictive of effects observed in human beings. Therefore, results obtained using compositions of this invention in these murine systems are representative of effects observed in human beings affected by Chlamydia or Helicobacter.

### Example 5

### Sample Collection and MOMP-Specific IgG and IgA ELISA Analysis I

One week following the final immunization of animals immunized according to Example 4 above, vaginal lavage (VL) was collected by flushing the vaginal vault with 50 ul of sterile PBS. Blood was collected by cardiac bleed following administration of a lethal dose of Sodium pentobarbitone.

MOMP-specific IgG and IgA in serum and VL, were determined by ELISA. Greiner immunopure ELISA plates (Interpath Ltd, Australia) were coated with C. muridarum MBP-MOMP (2 μg/well) diluted in borate-buffered solution (pH 9.6) and incubated overnight at 4° C. Plates were washed three times with 0.05% Tween 20 in PBS (PBST) and blocked for 1 hour at 37° C. with 100 μl of PBST containing 5% foetal calf serum. Plates were washed three times in PBST, and 100 μl of sample was added in duplicate and serially diluted two fold in PBST. Serum was diluted from 1/100 to 1/12,800 in PBST and VL fluid was diluted from 1/20 to 1/2,560. Sterile PBS was used as a negative control for each ELISA. Plates were covered and incubated at 37° C. for one hour and then washed three times with PBST. MOMP-bound antibodies were detected using an HRP-conjugated-anti-IgA or anti-IgG diluted 1/500 and 1/1,000 respectively (Southern Biotechnology Associates, Birmingham, Ala.), followed by a tetramethylbenzidine (TMB) colour-development system. The end point titer (E.P.T) value was defined as the mean of the PBS control wells+two standard deviations. Antigen specific antibody ratios were calculated by dividing E.P.T for ‘test’ group immunized samples by the E.P.T of non-immunized controls.

### Example 6

### T-Cell Proliferation and Cytokine Production I

Splenic lymphocytes were prepared as described from animals treated as in Example 1 above, labelled with CFSE then suspended at 5×10⁶ cells per ml in complete RPMI (RPMI 1640 supplemented with 5% FCS, L-Glutamine, 5×10⁻⁵ M 2-mercaptoethanol, HEPES buffer, penicillin-streptomycin, all from Trace Biosciences). Cells (100 μl) were added in triplicate to 96 well plates (unstained cells were used as a negative control). Media (background control), antigen MOMP (2 μg/well) or Con A (2 μg/well) (positive control) was added to appropriate wells. Plates were incubated at 37° C. in 5% CO₂ for 96 hours then the cells were collected by centrifugation. Cells were stained using a PECy7 pre-conjugated CD3 antibody (Becton Dickinson) and proliferating T cells were analysed using a FACSCanto flow cytometer (Becton Dickinson, Sydney, Australia). The percent of T cells induced to proliferate (>3 cell divisions) by in vitro culture with antigen was determined using Weasel software (Walter and Elisa Hall Institute, Melbourne, Australia).

### Example 7

### MOMP-Specific T-Cell Responses I

T cell proliferation was assayed by dye dilution assay using CFSE and is expressed as the percent of CD3+ cells that had undergone >3 cell divisions. In vitro re-stimulation of cells from mice immunized with MOMP+CT/CpG resulted in 10.2% (range 7-13%) of cells undergoing >3 rounds of division while 9.7% (range 8-11%) of CD3+ splenocytes from animals immunized with MOMP in Lipid C proliferated. Immunization with both MOMP and CT/CpG combined in Lipid C resulted in 9.9% (range 8-11%) of CD3 T cells proliferating following in vitro stimulation (Table 1).

TABLE 1

| | Non | | MOMP + | | Lipid C + | | Lipid C + MOMP + | |
|---|---|---|---|---|---|---|---|---|
| | Immunized | | CT/CpG | | MOMP | | CT/CpG | |
| Proliferation | 5% ± 2.9% | | 10.2% ± 4.2% | | 9.7% ± 2.5% | | 9.9% ± 2.7% | |
| IFNγ | 256 | (6-1111) | 6613 | (3491-10100) | 783 | (158-1487) | 4896 | (311-21629) |
| IL-12 | 33 | (12-42) | 100 | (37-170) | 53 | (14-109) | 56 | (33-86) |
| IL-4 | 0 | (0-2) | 84 | (35-129) | 0 | (0-1) | 5 | (0-20) |
| IL-10 | 144 | (25-313) | 499 | (77-819) | 236 | (7-486) | 260 | (39-572) |

As can be seen from Table 1, interferon gamma (IFNγ) was the predominant cytokine produced by cells from all experimental groups with higher levels seen in animals immunized with the CT/CpG adjuvants compared to those immunized with MOMP in Lipid C. The highest production of the Th2 cytokines IL-4 and IL-10 was also seen in cells from animals immunized with MOMP plus CT/CpG. Small increases in IL-10 and IL-12 production were seen in cells from all experimental groups, compared to non-immunized controls.

These results indicate that mice infected with Chlamydia have immunological reactions (e.g., T-cell proliferation and IFNγ production) similar to those observed in human beings exposed to this organism. These results also indicate that results observed in this murine system are predictive of effects to be observed in human beings.

### Example 8

### MOMP-Specific Antibodies I

One week following the final immunization MOMP-specific antibodies were detected in serum and vaginal lavage (FIG. 1). FIG. 1A depicts a graph of serum IgG antibodies were highest in animals immunized with MOMP+CT/CpG (EPT ratio>30, p<0.05 compared to non-immunized controls) and were also significantly increased in animals immunized with both MOMP and CT/CpG incorporated in Lipid C (EPT ratio>20, p<0.05). A 5-fold increase in serum IgG levels was seen in animals immunized with MOMP in Lipid C (FIG. 1A).

Vaginal lavage (VL) fluid collected from MOMP+CpG/CT immunized mice also showed increased IgG levels, with a statistically significant 10-fold increase compared to non-immunized controls (p<0.05). Additionally, lipid C and MOMP together produced a 2-fold increase in IgA was observed in VL fluid compared to non-immunized controls (FIG. 1B).

These results indicate that immunization using compositions of this invention were effective in eliciting an antibody response (e.g., increased IgG production). We conclude that lipid C can increase the immunological response of the vaginal mucosa to Chlamydia MOMP antigen. These results are predictive of effects seen in human beings infected with Chlamydia.

### Example 9

### Methods for C. muridarum Genital Challenge and Bacterial Recovery

Seven days before intravaginal challenge all mice treated either according to Example I or controls, received 2.5 mg of medroxyprogesterone acetate (Depo-Ralovera; Kenral, Rydalmere, New South Wales, Australia) subcutaneously. Mice were anaesthetized with xylazine (90 mg/kg) and ketamine (10 mg/kg) and challenged intravaginally with 5×10⁴ ifu of C. muridarum in 20 μl sucrose phosphate glutamate (SPG). Infection was allowed to progress for 21 days. Clearance of chlamydial infection was monitored by the collection of vaginal swabs (nasopharyngeal Calgiswab, Interpath), moistened with cold sterile SPG, at 3-day intervals from day 0 to 18 of infection. Swabs were placed into a sterile Eppendorf tube containing of 500 μl sterile SPG and two glass beads, vortexed and then stored at −80° C. Bacterial recovery was assessed using in vitro cell culture on McCoy cell monolayers as described (Barker C J, et al., Vaccine 2008 Mar. 4; 26(10):1285-96) and adapted (Hickey, D. K., et al., Vaccine 2002 22:4306-4315).

Briefly, McCoy cells were grown to 70% confluency in 48-well flat-bottom plates in complete DMEM (5% FCS, HEPES buffer, 5 μg/ml gentamicin, and 100 μg/ml streptomycin). 20 μl of serum or vaginal lavage fluid were incubated with 1000 inclusion-forming units (IFUs) of C. muridarum (C. trachomatis mouse pneumonitis biovar, ATCC VR-123) elementary bodies (EBs) for 30 min at 37° C. The antibody and C. muridarum solution was added to McCoy cells grown in complete DMEM (final volume 250 μl) and incubated for 3 h at 37° C. in 5% CO2. Media was removed and fresh DMEM (500 μl) containing 1 μg/ml cyclohexamide (Sigma-Aldrich, Castle Hill, Australia) was added to each well, followed by overnight incubation at 37° C. in 5% CO₂. Plates were observed by light microscopy for presence of Chlamydial inclusion bodies, at which point cells were washed two times in PBS then fixed for 10 min in 100% methanol, followed by Chlamydia-specific staining

Statistical Analysis. Data are presented as the mean±standard error of the mean (SEM). One-way analysis of variance (ANOVA) followed by Bonferroni's post-test was used to examine the differences between immunoglobulin concentrations and in vitro neutralization activity for each group. The significance level was set at P<0.05 for all tests. All statistical tests were performed using GraphPad Prism version 4.00 for Windows (GraphPad Software, San Diego Calif., USA.

### Example 10

### Protection Against Chlamydial Genital Challenge I

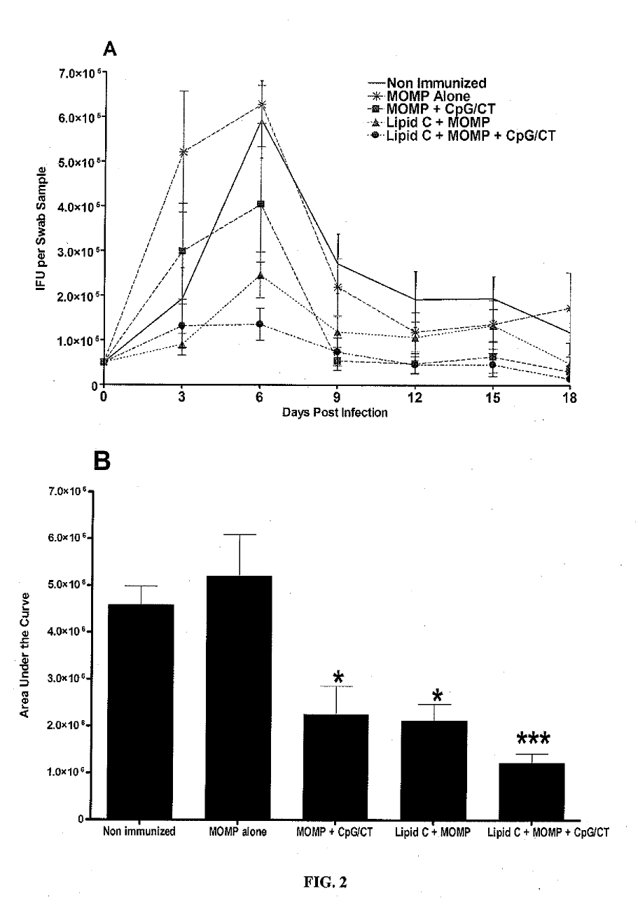

Bacterial shedding following live bacterial challenge was determined through in vitro culture of vaginal swabs collected at 3-day intervals (FIG. 2). FIG. 2A shows time-course of effects of recovery of infection forming units (IFU) following challenge with C. muridarum in immunized animals and in control animals.

FIG. 2B shows a graph of total infectivity is determined by measuring the total area under each curve. Oral immunization with MOMP alone did not significantly differ from non-immunized controls. Oral immunization with MOMP and CpG/CT resulted in a 30% reduction in bacterial shedding at the peak of infection (day 6; FIG. 2A). Over the 18-day infection period immunization with MOMP and CpG/CT resulted in a 50% reduction in infectivity compared to non-immunized controls (FIG. 2B).

Incorporating MOMP into Lipid C resulted in a 50% reduction in total infectivity (FIG. 2B, p<0.05) and a 60% reduction in bacterial shedding at day 6 (FIG. 2A). The greatest level of genital protection was observed following oral immunization with Lipid C formulated MOMP co-administered with CpG/CT. This group showed a 75% reduction in infection overall and a 75% reduction in C. muridarum recovered at day 6 (p<0.01). Oral immunization resulted in partial protection against infection in all groups. However, incorporation of both MOMP and CT/CpG in Lipid C significantly increased protection over that seen in animals immunized with MOMP alone in Lipid C or MOMP plus CT/CpG. Unexpectedly, addition of lipid C to compositions containing MOMP and CpG/CT further decreased recovery of Chlamydia, indicating that there is synergy between the prior art adjuvants CpG/CT and lipid C.

These results indicate that immunizing animals using compositions of this invention can significantly reduce infection caused by subsequent inoculation by Chlamydia. Further, these results indicate that substantial immunological protection against infection by Chlamydia can be obtained using lipid compositions of this invention without the need for the toxic adjuvants, CpG or CT. These results are predictive of effects observed in human beings, and represent a major, unexpected advantage over prior immunogenic compositions containing Chlamydial antigens.

### Example 11

### Collection of Samples for Analysis II

Samples of serum, vaginal lavage fluid, broncho-alveolar lavage fluid and fecal pellet washes were obtained 1 week post final immunization serum, vaginal lavage (VL) and broncho-alveolar lavage (BAL) was collected for chlamydial studies and serum and fecal pellet (FP) washes were collected for the H pylori studies. Terminal blood collection from heart was performed under lethal dose of sodium pentabarbitone using a sterile 23 gauge needle and a 1 ml syringe, blood was then transferred to a sterile 1.5 ml Eppendorf tube and serum obtained via centrifugation. Vaginal lavage fluid was collected by flushing the vaginal vault with 50 μl of sterile phosphate buffered saline (“PBS”). The fluid was collected into sterile 0.5 ml Eppendorf tubes. Broncho-alveolar lavage fluid was collected via inserting a blunted 23 gauge needle into the trachea, and the lungs were flushed twice with 750 μl Hanks Balanced Salt Solution (“HBSS”) and collected into a sterile 1.5 ml Eppendorf tube. Serum, VL and BAL samples are store at −20° C.

Two fresh fecal pellets were collected into 1 ml of sPBS containing 1 μg/ml soybean trypsin inhibitor and vortexed for 15 minutes then centrifuged at 10,000 rpm for 10 mins to remove solids. Supernate (800 μl) was added to fresh Eppendorf tubes containing 200 μl glycerol+200 μl PMSF (200 mM, Sigma), vortexed briefly and stored at −80° C.

### Example 12

### Methods for Measuring Antigen Specific IgG and IgA ELISA II

Levels of antigen specific IgG and IgA in serum, VL, BAL and FP were determined by enzyme-linked immunosorbent assays (“ELISA”). Greiner Immunopure™ ELISA plates (Interpath Ltd, Australia) were coated with either C. muridarum MBP-MOMP (2 μg/well) or H pylori crude sonicate (0.05 μg/well) depending on the immunization model, diluted in borate-buffered solution (pH 9.6) and incubates overnight at 4° C. Plates were washed three times with 0.05% Tween 20 in PBS (“PBST”) and blocked with 100 μl of PBST containing 5% foetal calf serum for the Chlamydia studies or 5% skim milk in PBST for the H pylori studies for 1 hour at 37° C. Plates were washed three times in PBST, and 100 μl of sample was added to row A in duplicate and serially diluted two fold seven times in PBST. Serum was diluted from 1/100 to 1/12,800 in PBST. VL fluid was diluted from 1/20 to 1/2,560. BAL fluid and FP washes were added neat and diluted to 1/128. Sterile PBS was used as a negative control for each ELISA. Plates were covered and incubated at 37° C. for one hour and then washed three times with PBST. MOMP-bound antibodies were detected using a HRP-conjugated anti-IgA or anti-IgG diluted 1/500 and 1/1,000 respectively (Southern Biotechnology Associates, Birmingham); followed by a tetramethylbenzidine (“TMB”) colour-development system (Hickey, D. K., et al., Vaccine, 2004. 22(31-32): p. 4306-15). The end point titer (E.P.T) cut off line was determined to be the mean of the PBS control wells+two standard deviations of the PBS. Antigen specific antibody ratios were calculated by dividing E.P.T for ‘test’ group immunized samples by the E.P.T of non-immunized controls.

### Example 13

### Spleen T Cell Proliferation Assay

A single cell suspension of splenocytes was prepared by homogenizing whole tissue through stainless steel sieves and wash twice in HBSS. Red blood cells were lysed by addition of red cell lysis buffer (NH₄Cl) and washed twice in HBSS. Cells were resuspended at 10⁷/ml in sterile PBS containing CFSE (5 μM final concentration) and incubated for 10 mins at 37° C. in the dark. CFSE was quenched by the addition of two volumes of FCS and washed three times in complete RPMI (RPMI 1640 base supplemented with 5% FCS, L-Glutamine, 5×10⁻⁵ M 2-mercaptoethanol, HEPES buffer, penicillin-streptomycin, all from Trace Biosciences).

Cells are resuspended at a density of 5×10⁶ cells/ml and 100 μl was added in triplicate to 96 well plates (unstained cells were used as a negative control). Media (background control), antigen (Chlamydia MOMP 2 μg/well or H pylori crude sonicate 0.1 μg/well) or Conconavalin A (“Con A”; 2 μg/well) (as a positive control) was added to appropriate wells. Plates were incubated at 37° C. in 5% CO₂ for 96 hours then the cells were collected by centrifugation. Cells were stained using a PerCy7 pre-conjugated CD3 antibody and positive cells were analysed at a fluorescence of 488 nm to identify CFSE labelled T cells using a FACSCanto™ flow cytometer (Becton Dickinson, Sydney, Australia). The percent of T cells induced to proliferate (>3 cell divisions) by in vitro culture with antigen was determined using Weasel™ software (Walter and Elisa Hall Institute, Melbourne, Australia).

### Example 14

### Methods for Assaying Cytokine Expression II

A single cell suspension of splenocytes was prepared as described above. Cells were resuspended at a density of 5×10⁶ cells/ml in complete RPMI and 100 μl was added in triplicate to 96 well plates (Greiner-Interpath Ltd). Media (background control), antigen (chlamydial MOMP 2 μg/well or H pylori crude sonicate 0.1 μg/well) or Con A (2 μg/well) (positive control) was added to appropriate wells and incubated at 37° C. in 5% CO₂ for 72 hours. Supernates were collected into a fresh Eppendorf tube and stored at −80° C. until cytokine analysis by Bioplex analysis. Bio-rad 6-plex mouse cytokine kit (Bio-Rad) identified concentrations (pg/ml) of IFNγ, TNFα, Interleukin-12, Interleukin-4, Interleukin-10 and granulocyte macrophage colony stimulating factor (“GMCSF”) in culture supernates and analysed on a Bioplex™ machine (BIO-RAD) according to the manufacturer's instructions.

### Example 15

### Helicobacter pylori SS1 Challenge and Bacterial Recovery

H pylori SS1 was grown in brain heart infusion (“BHI”) broth culture (Oxoid) containing 5% (v/v) horse serum and Skirrow's supplement for 2 days at 37° C. with 10% CO₂ and 95% humidity. Bacteria were pelleted by centrifugation at 300 rpm for 20 mins and resuspended at a concentration of 10⁸ cfu/ml. Mice were inoculated intragastrically 2 times over a 3 day period with 100 μl of bacterial suspension (˜10⁷ cfu/mouse) using a gavage needle under light isofluorothane anaesthetic. Animals were challenged 1 week after the final immunization and infection was allowed to progress for 6 weeks. After sacrifice the stomach was excised, cut along the greater curvature, and rinsed in saline to remove contents. The fundus was removed and stomachs were cut in half along the lesser curvature. One half of tissue was weighed and placed in 500 μl BHI. Stomach tissue was homogenised using a Tissue Tearor™ (Biospec Products Inc.) and 10-fold serial dilutions were prepared in BHI. 100 μl of each dilution (neat to 1:1000) was spread on CSA blood agar plates (as above) supplemented with Glaxo Selective Supplement A (“GSSA”, vancomycin 10 μg/ml, polymyxin B 0.33 μg/ml, bacitracin 20 μg/ml, nalidixic acid 1.2 μg/ml and amphoteracin B 5 mg/ml). After 6 days of incubation under humidified, microaerophilic conditions at 37° C., colonies were counted and colony forming units (“cfu”) per gram of stomach tissue was calculated. H pylori specific polymerase chain reaction (“PCR”) confirmation was also determined. Homogenised tissue (20 mg) was extracted using a DNA Wizard™ extraction kit (Promega) according to the manufacturer's instructions. PCR amplification of DNA (200 reaction) using GoTaq Green Master Mix™ (Promega, Australia) and helicobacter specific primers Hp 001 (5′ TATGACGGGTATCCGGC 3′; SEQ ID NO:2) and Hp 002 (5′ ATTCCACTTACCTCTCCCA 3′; SEQ ID NO:3) (sequence kindly supplied by Dr Sutton, Melbourne University). Amplification conditions were 95° C. for 2 minutes, followed by 30 cycles 94° C. 2 seconds each, 53° C. 2 seconds each, 72° C. 30 seconds each and a final step of 72° C. for 5 mins. Bands were visualised under UV light on a 1.5% agarose gel containing ethidium bromide.

### Example 16

### C. muridarum Genital Challenge and Bacterial Recovery

Seven days before intravaginal challenge, all mice received 2.5 mg of medroxyprogesterone acetate (Depo-Ralovera; Kenral, Rydalmere, New South Wales, Australia) subcutaneously. Mice were anaesthetized intraperitoneally with xylazine (90 mg/kg) and ketamine (10 mg/kg) and challenged intravaginally with 5×10⁴ infectious forming units (“IFU”s) of C. muridarum in 20 μl sucrose phosphate glutamate (“SPG”). Infection was allowed to progress for 21 days. Monitoring of clearance was observed through the collection of vaginal swabs (nasopharyngeal Calgiswab™ (Interpath) moistened with cold sterile SPG) at 3-day intervals from day 0 to 18 of infection. Each swab was placed into a sterile Eppendorf tube containing of 500 μl sterile SPG and two glass beads stored at −80° C.

Bacterial recovery was assessed using an in vitro cell culture. McCoy cell monolayers were grown to 70% confluence, 10 μl of vortexed swab solution was added to a culture well containing 300 μl of fresh DMEM containing 5% FCS, hepes buffer, gentamicin (5 μg/ml), and streptomycin (100 μg/ml). After a 3 hour incubation period at 37° C. 5% CO₂, medium was removed and replaced with 500 μl fresh complete DMEM containing 1 μg/ml (Sigma-Aldrich, Castle Hill, Australia) and incubated overnight at 37° C. 5% CO₂. Inclusion bodies were visualised under light microscopy, at which point cells and fixed for 10 mins in 100% methanol and stained using chlamydial specific staining as per Hickey at al. (Hickey, D. K., et al., Vaccine, 2004. 22(31-32): p. 4306-15).

### Example 17

### C. muridarum Respiratory Challenge and Bacterial Recovery

For respiratory challenge, animals were anaesthetized under light isofluorothane, and 10³ IFU of C. muridarum in cold sucrose phosphate glutamate (“SPG”) solution was administered via intranasal inoculation (5 μl each nare). The mice were then returned to their cages and housed under biosafety PC2 conditions, and infection was allowed to progress for 12 days (the time to estimated peak infection). After sacrifice, left weighed lung tissue was collected into 500 μl SPG containing two glass beads. The tissue was finely chopped with scissors and vortexed for 1 minute. 5 mg of tissue was added to 48 well culture plates containing McCoy cell monolayers grown to 70% confluencey, containing 500 μl of complete DMEM (5% FCS, hepes buffer, 5 μg/ml gentamicin, and 100 μg/ml streptomycin) and incubated at 37° C. 5% CO₂ for 3 hours. Mecium was removed and replaced with 500 μl fresh complete DMEM containing 1 μg/ml (Sigma-Aldrich, Castle Hill, Australia) and incubated overnight at 37° C. 5% CO₂. Inclusion bodies were visualized under light microscopy, at which point cells fixed for 10 mins in 100% methanol and stained using chlamydial specific staining as per Hickey at al. (Hickey, D. K., et al., Vaccine, 2004. 22(31-32): p. 4306-15).

### Example 18

### Statistical Analysis and Results

Data are presented as the mean±standard error of the mean (SEM) for the number of 5 mice in each experimental group. One-way analysis of variance (ANOVA) followed by Bonferroni's post-test was used to examine the differences between immunoglobulin concentration, ASC number, and neutralization ability for each group. The significance level was set at P<0.05 for all tests. All statistical tests were performed using GraphPad Prism™ version 4.00 for Windows (GraphPad Software, San Diego Calif., USA, www.graphpad.com).

### Example 19

### T Cell Responses II

T cell proliferation was measured using the CFSE dye dilution assay. Proliferation is presented as the percentage (%) of CD3 positive cells that had undergone >3 divisions. Three rounds of cell division were considered to be the threshold for background proliferation as MOMP and H pylori sonicate alone causes a low level of cell division in naive cells.

Following oral immunization with killed H. pylori only spleen T cells isolated from mice immunized with lipid C formulated with killed whole-cell H pylori admixed with CpG/CT showed increased division (6-7%) compared to non-immunized controls (1-2%). Cellular proliferation increased in all immunization groups following oral immunization with MOMP antigen compared to non-immunized controls. In vitro re-stimulation of cells from animals immunized with MOMP+CpG/CT resulted in 7-13% of cells undergoing >3 rounds of proliferation and animals immunized with MOMP+lipid C resulted in proliferation of 7-11% of cells. Combining both the adjuvants and lipid C with MOMP antigen resulted in 8-11% T cell proliferation. This combination did not further enhance T cell proliferation over levels seen in cells from animals immunized with MOMP+CpG/CT or lipid C formulated MOMP alone (Table 2).

The production of cytokines following in vitro stimulation with MOMP or H pylori sonicate was determined using Bioplex analysis. The predominant cytokine produced by T cells for all groups in both immunization models was IFNγ. The concentration of IFNγ produced by cells following immunization with the CpG/CT adjuvant was generally enhanced compared to that seen in cells from animals immunized with lipid C. IL-4 levels were uniformly low (<10 pg/ml) following immunization with killed whole-cell H pylori, whilst a result>100 pg/ml was observed in the chlamydial studies following immunization with MOMP admixed with CpG/CT. Generally IL-10 levels from all the immunization groups increased over levels seen in cultures of non-immunized controls. However, IL-10 production varied between experiments when MOMP or killed whole-cell H pylori antigens was formulated into lipid C. Increased production of IL-12 following in vitro antigen stimulation was observed in cultures of cells from all immunization groups, although a variation between experiments was observed in the lipid C formulated antigen alone group for both models (Table 2). IFNγ was the dominant cytokine

Table 2 depicts antigen specific splenic T cell proliferation and cytokine expression was determined in vitro one week after final immunization.

TABLE 2
In vitro Antigen Specific Splenic T cell
Proliferation & Cytokine Production

| | Non | Killed HP + | LipoVax + | LipoVax + Killed |
|---|---|---|---|---|
| | Immunized | CpG/CT | Killed HP | HP + CpG/CT |
| Proliferation | | | | |
| Experiment1 | 1.68 | 2.04 | 0.00 | 6.07 |
| Experiment2 | 1.91 | 4.47 | 3.09 | 6.98 |
| IFNγ | | | | |
| Experiment1 | 0 | 312.45 | 105 | 28.25 |
| Experiment2 | 4.72 | 719.25 | 39.38 | 140.4 |
| IL-12 | | | | |
| Experiment1 | 18.6 | 56.15 | 62.1 | 34.7 |
| Experiment2 | 28.62 | 80.48 | 19.2 | 60.47 |
| IL-4 | | | | |
| Experiment1 | 0 | 11 | 0.05 | 0 |
| Experiment2 | 0 | 8.01 | 0 | 4.69 |
| IL-10 | | | | |
| Experiment1 | 132.3 | 246.2 | 269.5 | 165.4 |
| Experiment2 | 174.27 | 563.27 | 109.79 | 404.94 |

| | Non | MOMP + | LipoVax + | LipoVax + |
|---|---|---|---|---|
| | Immunized | CpG/CT | MOMP | MOMP + CpG/CT |
| Proliferation | | | | |
| Experiment1 | 8.12 | 13.22 | 11.60 | 11.86 |
| Experiment2 | 2.91 | 7.26 | 7.94 | 8.02 |
| IFNγ | | | | |
| Experiment1 | 64.25 | 10099.6 | 1487.25 | 962.9 |
| Experiment2 | 1111.14 | 6252.81 | 157.97 | 21628.78 |
| IL-12 | | | | |
| Experiment1 | 42.45 | 170.45 | 109.05 | 82.2 |
| Experiment2 | 42.94 | 124.6 | 35.12 | 86.22 |
| IL-4 | | | | |
| Experiment1 | 0 | 129.5 | 0.05 | 0.1 |
| Experiment2 | 2.8 | 68.51 | 0 | 20.04 |
| IL-10 | | | | |
| Experiment1 | 190.8 | 801.05 | 486.5 | 343.85 |
| Experiment2 | 313.02 | 819.76 | 203.9 | 572.27 |

Oral immunization induced significant MOMP specific proliferation of T cells isolated from spleen one week after final immunization in all groups. Quantitative cytokine production was determined through Bioplex analysis and results are represented as pg/ml. Results are representative of two separate experiments.

These results indicate that after infection by H pylori or Chlamydia, mice responded with a typical immunological reaction, with T-Cell proliferation and increased production of inflammatory mediators. Additionally, immunization using compositions of this invention were effective in increasing the immunological response to inoculation with H pylori or Chlamydia. These results also indicate that substantial immunological protection can be elicited using compositions of this invention without the use of the toxic CpG or CT adjuvants. These results are predictive of effects observed in human beings and represent an unexpected improvement over prior art compositions.

### Example 20

### Helicobacter Specific Antibodies

Following oral immunization of mice with killed whole-cell H pylori, the production of antigen specific antibodies in serum and fecal pellet (FP) where detected using H pylori crude cell sonicate coated ELISA plates. Oral immunization led to the production of systemic IgG and fecal IgA H pylori antibodies (FIG. 3). A significant increase was observed compared to non-immunized controls following immunization with killed whole-cell H. pylori admixed with CpG/CT resulting in a 4-fold (p<0.05) and 6-fold increase in serum IgG and fecal IgA respectively. The addition of lipid C reduced both systemic and gastric mucosal antibody production by about 50%. Animals receiving either lipid C formulated killed whole-cell H pylori alone showed a 2-fold increase of serum IgG and a 3-fold increase in fecal IgA (p<0.05) compared to non-immunized controls. Lipid C formulation of killed whole-cell H pylori admixed with CpG/CT also resulted in a 2-fold increase of serum IgG and a 3-fold increase in fecal IgA, however this did not statistically differ from non-immunized controls.

### Example 21

### Chlamydia and H. pylori Specific Antibodies II

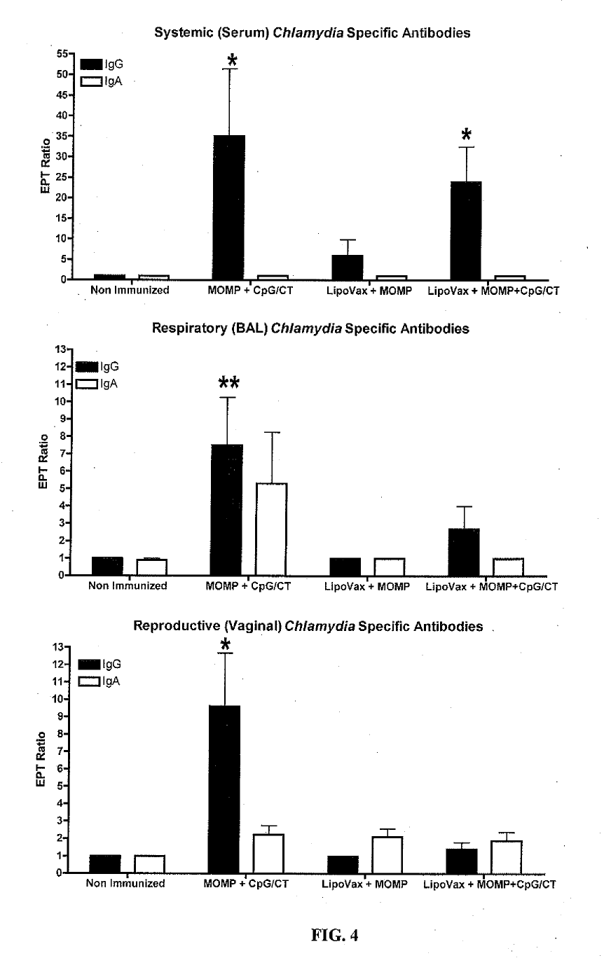

MOMP specific antibodies were detected one week following final immunization in serum, broncho-alveolar lavage (BAL) and vaginal lavage (VL) samples. Significant systemic IgG production was induced following immunization with killed whole-cell H. pylori admixed with CpG/CT compared to non-immunized controls (p<0.05). Lipid C formulated immunization solution also significantly increased systemic IgG compared to non-immunized controls (FIG. 4A). An increased production of both IgG (7-fold, p<0.01) and IgA (5-fold) antibodies compared to non-immunized controls in respiratory BAL wash was observed from mice that received unformulated MOMP admixed with CpG/CT orally. A 3-fold increase was observed when MOMP+CpG/CT was formulated with lipid C and no antibody production was observed in BAL fluid collected from mice immunized with lipid C formulated MOMP alone (FIG. 4B). VL fluid collected from MOMP+CpG/CT immunized mice also contained increased IgG levels with a significant 10-fold increase compared to non-immunized controls (p<0.05). Additionally, a 2-fold increase in IgA was observed in VL fluid for all immunization groups compared to non-immunized controls (FIG. 4C) although this was not statistically significant.

### Example 23

### Gastrointestinal Tract Protection

Following oral immunization it was expected that adaptive immune responses would be elicited locally in the gut. However, immunizing mice with killed whole-cell H pylori antigen and the potent mucosal adjuvant CpG/CT resulted in a reduction in bacterial colonization. Incorporating the killed whole-cell H. pylori antigen alone into a lipid C matrix to protect antigens from digestive enzymes again did induce a degree of protection compared to non-immunized controls. Only through oral feeding of lipid C incorporated killed whole-cell H pylori with CpG/CT was a statistically significant reduction in colonization observed (p<0.05). In this immunization group a 1 log reduction in live H pylori SS1 bacteria was recovered compared to non-immunized controls (FIG. 5).

Table 3 below presents data on the numbers of animals having detectable infection by H. pylori after innoculation with the compositions indicated.

TABLE 3
Numbers of Animals Having Detectable Infection

| | Treatment | | | |
|---|---|---|---|---|
| | | | | Lipid C + |
| | Non- | Killed HP + | Lipid C + | Killed HP + |
| | Immunized | CpG/CT | Killed HP | CpG/CT |
| Infected mice | 8/10 | 7/10 | 9/10 | 4/10 |

From FIG. 5, it is apparent that the recovery of viable H pylori from animals treated with lipid C and H pylori antigens is reduced compared to either control animals or animals exposed to H pylori antigen alone, thereby indicating a decrease in bacterial load in vaccinated animals. Immunization with lipid C and H. pylori antigen together did not provide complete protection, as there was recovery of viable H. pylori organisms, and the mice so treated showed signs of infection (Table 3).

These results indicate that oral immunization using lipid C and heat killed antigens from H. pylori can be effective in decreasing gastric infection by decreasing the bacterial load in the affected tissue. Furthermore, the finding that adding lipid C to orally administered compositions containing H pylori antigens and CpG/CT further reduced bacterial recovery indicated that lipid C and the prior art adjuvants CpG/CT can act synergistically to provide unexpectedly enhanced mucosal immunity against H pylori.

### Example 24

### Respiratory Tract Protection

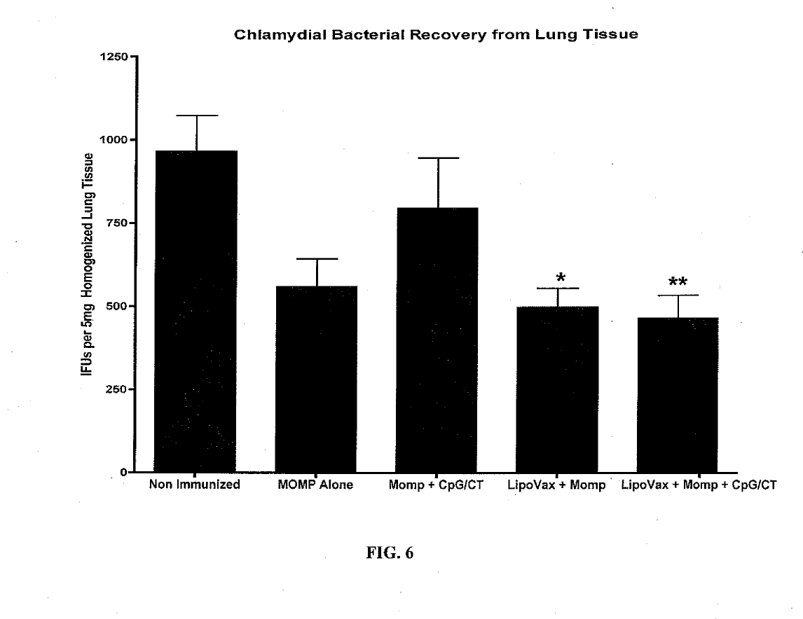

Chlamydia was isolated from homogenised lung tissue at peak of infection (day 12) and infection forming units determined through in vitro cell culture using methods described herein. Results are shown in FIG. 6. We found no significant difference in Chlamydia recovery following immunization of MOMP+CpG/CT compared to non-immunized controls. However, we observed statistically significant protection of the respiratory tract after immunization with orally administered lipid C+MOMP+CpG/CT or lipid C+MOMP. In these immunization groups a 50% reduction in bacteria recovered was observed compared to non-immunized controls was observed (p<0.05; n=10 animals in each group).

These results indicate that immunization with compositions of this invention can be effective in protecting animals from infection by Chlamydia. These unexpected findings indicate that it is now possible to provide effective immunological protection using compositions of this invention without the necessity for using toxic CpG or CT adjuvants.

Herein we demonstrated that immunogenic compositions containing long-chain fatty acids and non-infective antigens can be an effective delivery medium for the enhancement of protective mucosal immunity following oral immunization. In addition, lipid-containing compositions of this invention can be used in conjunction with ‘safe’ purified protein antigens to induce protection at the genital and respiratory mucosae. Use of lipid-containing immunogenic compositions of this invention for the oral administration of protein antigens potentially provides an inexpensive, easy to administer, safe alternative to live vaccines currently in use for human vaccination.

### REFERENCES

All references and sequence listings cited herein are expressly incorporated fully by reference as if separately so incorporated. Any differences in the citations in the text and in this section will be resolved with reference to the citations below.
1. Owen, R. and A. Jones, Epithelial cell specialization within human Peyer's patches: an ultrastructural study of intestinal lymphoid follicles. Gastroenterology, 1974. February; 66(2): p. 189-203.
2. Iwasaki, A. and B. Kelsall, Localization of distinct Peyer's patch dendritic cell subsets and their recruitment by chemokines macrophage inflammatory protein (MIP)-3alpha, MIP-3beta, and secondary lymphoid organ chemokine. J Exp Med, 2000. April 17; 191(8): p. 1381-94.
3. Giudice, E. L. and J. D. Campbell, Needle-free vaccine delivery, in Adv Drug Deliv Rev. 2006. Apr. 20; 58(1) p. 68-89.
4. Cross, M. L., B. M. Buddle, and F. E. Aldwell, The potential of oral vaccines for disease control in wildlife species, in Vet J. 2007. November; 174(3):472-80.
5. Faal, N., et al., Conjunctival FOXP3 expression in trachoma: do regulatory T cells have a role in human ocular Chlamydia trachomatis infection? PLoS Med, 2006. August; 3(8): p. e266.
6. Mabey, D. and R. Peeling, Lymphogranuloma venereum. Sexually transmitted infections, 2002. April; 78(2): p. 90-2.
7. Hansbro, P. M., et al., Role of atypical bacterial infection of the lung in predisposition/protection of asthma. Pharmacol Ther, 2004._March; 101(3): p. 193-210.
8. Horvat, J., et al., Neonatal Chlamydial Infection Induces Mixed T Cell Responses that Drive Allergic Airways Disease. Am J Respir Crit. Care Med, 2007. Sep. 15; 176(6):556-64.
9. Del Giudice, G., et al., The design of vaccines against Helicobacter pylori and their development. Annu Rev Immunol, 2001. 19: p. 523-63.
10. Frenck, R. W., Jr. and J. Clemens, Helicobacter in the developing world. Microbes Infect, 2003. July; 5(8): p. 705-13.
11. Enno, A., et al., Antigen-dependent progression of mucosa-associated lymphoid tissue (MALT)-type lymphoma in the stomach. Effects of antimicrobial therapy on gastric MALT lymphoma in mice. Am J Pathol, 1998. June; 152(6): p. 1625-32.
12. Correa, P., Bacterial infections as a cause of cancer. J Natl Cancer Inst, 2003. Apr. 2; 95(7): p. E3.
13. Ernst, P. and B. Gold, The disease spectrum of Helicobacter pylori: the immunopathogenesis of gastroduodenal ulcer and gastric cancer. Annu Rev Microbiol, 2000. 54: p. 615-40.
14. Uemura, N., et al., Helicobacter pylori infection and the development of gastric cancer. N Engl J Med, 2001. Sep. 13; 345(11): p. 784-9.
15. G. Mayrhofer, and L. Spargo, Distribution of class II major histocompatibility antigens in enterocytes of the rat jejunum and their association with organelles of the endocytic pathway Immunology May 1990. 70(1):11-19.
16. Hershberg, R. M., et al., Highly polarized HLA class II antigen processing and presentation by human intestinal epithelial cells, in J Clin Invest. 1998. Aug. 15; 102(4): p. 792-803.
17. Rescigno, M., et al., Dendritic cells express tight junction proteins and penetrate gut epithelial monolayers to sample bacteria. Nat Immunol, 2001. April; 2(4): p. 361-7.
18. Bockman, D., W. Boydston, and D. Beezhold, The role of epithelial cells in gut-associated immune reactivity. Ann N Y Acad Sci, 1983. Jun. 30; 409: p. 129-44.
19. Bockman, D. and M. Cooper, Pinocytosis by epithelium associated with lymphoid follicles in the bursa of Fabricius, appendix, and Peyer's patches. An electron microscopic study. Am J Anat, 1973. April; 136(4): p. 455-77.
20. Neutra, M. R., et al., Transport of membrane-bound macromolecules by M cells in follicle-associated epithelium of rabbit Peyer's patch, in Cell Tissue Res. 1987. March; 247(3): p. 537-46.
21. Holmgren, J., N. Lycke, and C. Czerkinsky, Cholera toxin and cholera B subunit as oral-mucosal adjuvant and antigen vector systems. Vaccine, 1993. September; 11(12): p. 1179-84.
22. Hickey, D. K., et al., Intranasal immunization with C. muridarum major outer membrane protein (MOMP) and cholera toxin elicits local production of neutralising IgA in the prostate. Vaccine, 2004. Oct. 22; 22(31-32): p. 4306-15.
23. Skelding, K. A., et al., Comparison of intranasal and transcutaneous immunization for induction of protective immunity against Chlamydia muridarum respiratory tract infection. Vaccine, 2006. Jan. 16; 24(3): p. 355-66.
24. Glenn, G., et al., Transcutaneous immunization with cholera toxin protects mice against lethal mucosal toxin challenge. J Immunol, 1998. Oct. 1; 161(7): p. 3211-4.
25. Yu, J., et al., Transcutaneous immunization using colonization factor and heat-labile enterotoxin induces correlates of protective immunity for enterotoxigenic Escherichia coli. Infect Immun, 2002. March; 70(3): p. 1056-68.
26. Berry, L. J., et al., Transcutaneous immunization with combined cholera toxin and CpG adjuvant protects against Chlamydia muridarum genital tract infection. Infect Immun, 2004. February; 72(2): p. 1019-28.
27. van Ginkel, F., et al., Cutting edge: the mucosal adjuvant cholera toxin redirects vaccine proteins into olfactory tissues. J Immunol, 2000. Nov. 1; 165(9): p. 4778-82.
28. Bangham, A. D. and R. W. Home, Negative staining of phospholipids and their structural modification by surface active agents as observed in the electron microscope, in J Mol. Biol. 1964. May; 8: p. 660-8.
29. Niikura, M., et al., Chimeric recombinant hepatitis E virus-like particles as an oral vaccine vehicle presenting foreign epitopes, in Virology. 2002. Feb. 15; 293(2): p. 273-80.
30. Guerrero, R. A., et al., Recombinant Norwalk virus-like particles administered intranasally to mice induce systemic and mucosal (fecal and vaginal) immune responses, in J. Virol. 2001. October; 75(20): p. 9713-22.
31. Szoka, F. and D. Papahadjopoulos, Procedure for preparation of liposomes with large internal aqueous space and high capture by reverse-phase evaporation, in Proc Natl Acad Sci USA. 1978. September; 75(9): p. 4194-8.
32. Deamer, D. and A. D. Bangham, Large volume liposomes by an ether vaporization method, in Biochim Biophys Acta. 1976. Sep. 7; 443(3): p. 629-34.
33. Chapman, C. J., et al., Effects of solute concentration on the entrapment of solutes in phospholipid vesicles prepared by freeze-thaw extrusion, in Chem Phys Lipids. 1991. December; 60(2): p. 201-8.
34. Sou, K., et al., Effective encapsulation of proteins into size-controlled phospholipid vesicles using freeze-thawing and extrusion, in Biotechnol Prog. 2003. September-October; 19(5): p. 1547-52.
35. Kirby, C. J. and G. Gregoriadis, Preparation of liposomes containing factor VIII for oral treatment of haemophilia, in Journal of Microencapsulation. 1984. January-March; 1(1): p. 33-45.
36. Erickson, R. H. and Y. S. Kim, Digestion and absorption of dietary protein, in Annu Rev Med. 1990. 41: p. 133-9.
37. Aldwell, F. E., et al., Oral delivery of Mycobacterium bovis BCG in a lipid formulation induces resistance to pulmonary tuberculosis in mice. Infect Immun, 2003. January; 71(1): p. 101-8.
38. Aldwell, F., et al., Oral vaccination with Mycobacterium bovis BCG in a lipid formulation induces resistance to pulmonary tuberculosis in brushtail possums. Vaccine, 2003. Dec. 8; 22(1): p. 70-6.
39. Colditz, G. A., et al., The efficacy of bacillus Calmette-Guérin vaccination of newborns and infants in the prevention of tuberculosis: mew-analyses of the published literature, in Pediatrics. 1995. July; 96(1 Pt 1): p. 29-35.
40. Colditz, G. A., et al., Efficacy of BCG vaccine in the prevention of tuberculosis. Meta-analysis of the published literature, in JAMA. 1994. Mar. 2; 271(9): p. 698-702.
41. Fine, P. E., Variation in protection by BCG: implications of and for heterologous immunity, in Lancet. 1995. Nov. 18; 346(8986): p. 1339-45.
42. Aldwell, F. E., et al., Oral delivery of lipid-encapsulated Mycobacterium bovis BCG extends survival of the bacillus in vivo and induces a long-term protective immune response against tuberculosis. Vaccine, 2006. Mar. 15; 24(12): p. 2071-8.
43. Su, H., et al., A recombinant Chlamydia trachomatis major outer membrane protein binds to heparan sulfate receptors on epithelial cells. Proc Natl Acad Sci USA, 1996. Oct. 1; 93(20): p. 11143-8.
44. Lee, A., et al., A standardized mouse model of Helicobacter pylori infection: introducing the Sydney strain. Gastroenterology, 1997. April; 112(4): p. 1386-97.
45. Sutton, P., J. Wilson, and A. Lee, Further development of the Helicobacter pylori mouse vaccination model. Vaccine, 2000. Jun. 1; 18(24): p. 2677-85.
46. Aldwell, F. E., et al., Oral vaccination of mice with lipid-encapsulated Mycobacterium bovis BCG: anatomical sites of bacterial replication and immune activity, in Immunol Cell Biol. 2005. October; 83(5): p. 549-53.
47. Buddle, B., et al., Effect of oral vaccination of cattle with lipid-formulated BCG on immune responses and protection against bovine tuberculosis. Vaccine, 2005. May 20; 23(27): p. 3581-9.
48. Fujinaga, Y., et al., Gangliosides That Associate with Lipid Rafts Mediate Transport of Cholera and Related Toxins from . . . , in Molecular Biology of the Cell. 2003 December; 14(12):4783-93.49. Orlandi, P. A. and P. H. Fishman, Filipin-dependent inhibition of cholera toxin: evidence for toxin internalization and activation through caveolae-like domains, in J. Cell Biol. 1998. May 18; 141(4): p. 905-15.
50. Wolf, A. A., Y. Fujinaga, and W. I. Lencer, Uncoupling of the cholera toxin-G(M1) ganglioside receptor complex from endocytosis, retrograde Golgi trafficking, and downstream signal transduction by depletion of membrane cholesterol, in J Biol. Chem. 2002. May 3; 277(18): p. 16249-56.
51. Triantafilou, M., et al., Mediators of innate immune recognition of bacteria concentrate in lipid rafts and facilitate lipopolysaccharide-induced cell activation, in J Cell Sci. 2002. Jun. 15; 115(Pt 12): p. 2603-11.
52. Triantafilou, M., et al., Lipoteichoic acid and toll-like receptor 2 internalization and targeting to the Golgi are lipid raft-dependent, in J Biol. Chem. 2004. Sep. 24; 279(39): p. 40882-9.
53. Dolganiuc, A., et al., Acute ethanol treatment modulates Toll-like receptor-4 association with lipid rafts, in Alcohol Clin Exp Res. 2006. January; 30(1): p. 76-85.
54. Lata, E., et al., TLR9 signals after translocating from the ER to CpG DNA in the lysosome, in Nat. Immunol. 2004. February; 5(2): p. 190-8.
55. Simons, K. and W. L. Vaz, Model systems, lipid rafts, and cell membranes, in Annual review of biophysics and biomolecular structure. 2004._{—}33: p. 269-95.
56. Dykstra, M., et al., Location is everything: lipid rafts and immune cell signaling, in Annu Rev Immunol. 2003. 21: p. 457-81.
57. Klausner, R. D., et al., Lipid domains in membranes. Evidence derived from structural perturbations induced by free fatty acids and lifetime heterogeneity analysis, in J Biol. Chem. 1980. Feb. 25; 255(4) p. 1286-95.
58. Stulnig, T. M., et al., Polyunsaturated fatty acids inhibit T cell signal transduction by modification of detergent-insoluble membrane domains. J Cell Biol, 1998. Nov. 2; 143(3): p. 637-44.
59. Stulnig, T. M., et al., Polyunsaturated eicosapentaenoic acid displaces proteins from membrane rafts by altering raft lipid composition, in J Biol. Chem. 2001. Oct. 5; 276(40): p. 37335-40.
60. Weatherill, A. R., et al., Saturated and polyunsaturated fatty acids reciprocally modulate dendritic cell functions mediated through TLR4. J Immunol, 2005. May 1; 174(9): p. 5390-7.
61. Challacombe, S. J. and T. B. Tomasi, Systemic tolerance and secretory immunity after oral immunization, in J Exp Med. 1980. Dec. 1; 152(6): p. 1459-72.
62. Caldwell, H. D., J. Kromhout, and J. Schachter, Purification and partial characterization of the major outer membrane protein of Chlamydia trachomatis, in Infect Immun. 1981. March; 31(3): p. 1161-76.
63. Charman, W., et al., Physiochemical and physiological mechanisms for the effects of food on drug absorption: the role of lipids and pH. Journal of pharmaceutical sciences, 1997. March; 86(3): p. 269-82.
64. Armand, M., Lipases and lipolysis in the human digestive tract: where do we stand? Current opinion in clinical nutrition and metabolic care, 2007. March; 10(2): p. 156-64.
65. Morein, B., et al., Iscom, a novel structure for antigenic presentation of membrane proteins from enveloped viruses, in Nature. 1984. Mar. 29-Apr. 4; 308(5958): p. 457-60.
66. Skene, C. D. and P. Sutton, Saponin-adjuvanted particulate vaccines for clinical use, in Methods. 2006. September; 40(1): p. 53-9.
67. Mishra, D., et al., Elastic liposomes mediated transcutaneous immunization against Hepatitis B, in Vaccine. 2006._May 29; 24(22):p 4847-55.
68. Wang, D., et al., Intranasal immunization with liposome-encapsulated plasmid DNA encoding influenza virus hemagglutinin elicits mucosal, cellular and humoral immune responses, in J Clin Virol. 2004. December; 31 Suppl 1: p. S99-106.
69. Perrie, Y., et al., Liposome (Lipodine)-mediated DNA vaccination by the oral route, in Journal of liposome research. 2002. February-May; 12(1-2): p. 185-97.
70. *dander, A., et al., Immune responses to ISCOM formulations in animal and primate models, in Vaccine. 2001. Mar. 21; 19(17-19): p. 2661-5.
71. Beagley K W, Timms P. Chlamydia trachomatis infection: incidence, health costs and prospects for vaccine development, in. Journal of Reproductive Immunology 2000; August; 48(1):47-68.
72. Westrom L, Mardh P A. Chlamydial salpingitis. Br Med Bull 1983 April; 39(2):145-50.
73. Ho J L, He S, Hu A, Geng J, Basile F G, Almeida M G, et al. Neutrophils from human immunodeficiency virus (HIV)-seronegative donors induce HIV replication from HIV-infected patients' mononuclear cells and cell lines: an in vitro model of HIV transmission facilitated by Chlamydia trachomatis. J Exp Med 1995 Apr. 1; 181(4):1493-505.
74. Kaul R, Nagelkerke N J, Kimani J, Ngugi E, Bwayo J J, Macdonald K S, et al. Prevalent herpes simplex virus type 2 infection is associated with altered vaginal flora and an increased susceptibility to multiple sexually transmitted infections, in J Infect Dis 2007 Dec. 1; 196(11):1692-7.
75. Stamm W E. Rationale for a Vaccine. In: Woodall J P, editor. Proceedings of the Chlamydia Vaccine Development Colloquium; 2004; Alexandria, Va.: The Albert B. Sabin Vaccine Institute; 2004. p. 15-8.
76. Weiner H L. Oral tolerance, an active immunologic process mediated by multiple mechanisms, in J Clin Invest 2000 October; 106(8):935-7.
77. Williams N A, Hirst T R, Nashar T O. Immune modulation by the cholera-like enterotoxins: from adjuvant to therapeutic. Immunol Today 1999 February; 20(2):95-101.
78. Nol P, Palmer M V, Waters W R, Aldwell F E, Buddle B M, Triantis J M, et al. Efficacy of oral and parenteral routes of mycobacterium bovis bacille Calmette-Guerin vaccination against experimental bovine tuberculosis in white-tailed deer (Odocoileus virginianus): a feasibility study. J Wildl Dis 2008 April; 44(2):247-59.
79. Clark S, Cross M L, Nadian A, Vipond J, Court P, Williams A, et al. Oral vaccination of guinea pigs with Mycobacterium bovis Bacille Calmette-Guerin (BCG) vaccine in a lipid matrix protects against aerosol infection with virulent M bovis. Infect Immun 2008 Jun. 2.
80. Barker C J, Beagley K W, Hafner L M, Timms P. In silico identification and in vivo analysis of a novel T-cell antigen from Chlamydia, NrdB. Vaccine 2008 Mar. 4; 26(10):1285-96.
81. Challacombe S J, Rahman D, O'Hagan D T. Salivary, gut, vaginal and nasal antibody responses after oral immunization with biodegradable microparticles. Vaccine 1997 February; 15(2):169-75.
82, Briese V, Pohl W D, Noack K, Tischner H, Waldman R H. Influenza specific antibodies in the female genital tract of mice after oral administration of live influenza vaccine. Arch Gynecol 1987; 240(3):153-7.
83. Zhang X, Lou Y H, Koopman M, Doggett T, Tung K S, Curtiss R r. Antibody responses and infertility in mice following oral immunization with attenuated Salmonella typhimurium expressing recombinant murine ZP3 [published erratum appears in Biol Reprod 1997 April; 56(4):1069]. Biology of Reproduction 1997; 56(1):33-41.
84. Whittum-Hudson J A, An L L, Saltzman W M, Prendergast R A, MacDonald A B. Oral immunization with an anti-idiotypic antibody to the exoglycolipid antigen protects against experimental Chlamydia trachomatis infection. Nature Medicine 1996; 2(10):1116-21.
85. Grayston J T, Wang S P. The potential for vaccine against infection of the genital tract with Chlamydia trachomatis. [Review][19 refs]. Sexually Transmitted Diseases 1978; 5(2):73-7.
86. Armand M. Lipases and lipolysis in the human digestive tract: where do we stand? Curr Opin Clin Nutr Metab Care 2007 March; 10(2):156-64.
87. Horejsi V. The roles of membrane microdomains (rafts) in T cell activation. Immunol Rev 2003 February; 191:148-64.
88. Anderson H A, Hiltbold E M, Roche P A. Concentration of MHC class II molecules in lipid rafts facilitates antigen presentation. Nat Immunol 2000 August; 1(2):156-62.
89. Mahoney E M, Hamill A L, Scott W A, Cohn Z A. Response of endocytosis to altered fatty acyl composition of macrophage phospholipids. Proc Natl Acad Sci USA 1977 November; 74(11):4895-9.
90. Weatherill A R, Lee J Y, Zhao L, Lemay D G, Youn H S, Hwang D R. Saturated and polyunsaturated fatty acids reciprocally modulate dendritic cell functions mediated through TLR4. J Immunol 2005 May 1; 174(9):5390-7.
91. Schweitzer S C, Reding A M, Patton H M, Sullivan T P, Stubbs C E, Villalobos-Menuey E, et al. Endogenous versus exogenous fatty acid availability affects lysosomal acidity and MHC class II expression. J Lipid Res 2006 November; 47(11):2525-37.
92. Calder P C, Bond J A, Newsholme E A. Fatty acid inhibition of concanavalin A-stimulated lymphocyte proliferation. Biochem Soc Trans 1989 December; 17(6):1042-3.
93. Calder P C, Bond J A, Newsholme E A. Fatty acid inhibition of lipopolysaccharide-stimulated B lymphocyte proliferation. Biochem Soc Trans 1990 October; 18(5):904-5.
94. Hickey, D. K, R. C. Jones, S, Bao, A. E. Blake, K A. Skelding, L. J. Berry and K. W. Beagley, Intranasal immunization with C. muridarum major outer membrane protein (MOMP) and cholera toxin elicits local production of neutralizing IgA in the prostate. Vaccine 2004 22:4306-4315.

We claim:

1. An immunogenic composition, comprising:
a lipid formulation containing at least 30% C₁₆ to C₁₈ fatty acids, said formulation having a solid to fluid transition temperature above about 30° C.; and
an antigenic component from Chlamydia or Helicobacter, said composition capable of eliciting a mucosal immune response in an animal receiving said composition via oral or gastrointestinal route.

2. The composition of claim 1, wherein said lipid formulation comprising about 1% myristic acid, about 25% palmitic acid, about 15% stearic acid, about 50% oleic acid and about 6% linoleic acid (“Lipid C”).

3. The composition of claim 1, said lipid component comprises Lipid C, Lipid K (7.6% caprylic acid (C8:0), 6.8% capric acid (C10:0), 45.1% lauric acid (C12:0), 18.3% myristic acid (C14:0), 9.7% palmitic acid (C16:0), 2.7% stearic acid (C18:0), 7.7% oleic acid (C18:1), and 2.3% linoleic acid (C18:2)), Lipid PK (7.0% caproic acid, 5.8% capric acid, 45.0% laurate, 18.2% myristic acid, 9.9% palmitic acid, 2.9% stearic acid, 7.6% oleic acid and 2.3% linoleic acid and a total saturated fat composition of 88.8%, a monounsaturated composition of 7.6%, and a polyunsaturated fat composition of 2.3%) or Lipid SPK (6.7% caproic acid, 5.6% capric acid, 44.3% laurate, 17.9% myristic acid, 9.6% palmitic acid, 3.0% stearic acid, 8.4% oleic acid and 2.6% linoleic acid, and a total saturated fat composition of 87.3%, a monounsaturated fat composition of 8.4%, and a polyunsaturated fat composition of 2.6%).

4. The composition of claim 1, where said antigenic component from said Chlamydia organism is one or more of a major outer membrane protein (MOMP), a 60 kDa-62 kDa cysteine-rich protein membrane protein, a 15 kDa cysteine-rich membrane protein, a 74 kDa species-specific protein, a 31 kDa eukaryotic cell-binding protein or a 18 kDa eukaryotic cell-binding protein.

5. The composition of claim 4, wherein said MOMP is a serotype selected from the group consisting of A, B, Ba, C, D, E, F, G, Hi, I, J, K, L1, L2 or L3.

6. The composition of claim 4, wherein said antigenic component of Helicobacter is whole killed antigen from H. pylori.

7. The composition of claim 1, further comprising an additional adjuvant.

8. The composition of claim 7, wherein said additional adjuvant is one or more of cholera toxin (CT) and CpG oligodeoxynucleotide (“CpG-ODN”: SEQ ID NO:1).

9. An oral immunogenic composition comprising MOMP and Lipid C.

10. A method for treating a mucosal infection caused by an organism of the family Chlamydiae, comprising administering the composition of claim 1 to an animal in need thereof.

11. The method of claim 10, wherein said treating occurs via induction of a mucosal immune response in said animal.

12. The method of claim 10, wherein said animal is a human being.

13. The method of claim 10, wherein said composition comprises MOMP, Lipid C and one or more of CT and CpG-ODN.

14. A method for providing immunological protection to an animal against infection caused by Chlamydia or Helicobacter, comprising:
administering a composition of claim 1 to said animal, wherein said animal has a finding indicative of an immune response.

15. The method of claim 14, wherein said finding is selected from the group consisting of thymocyte (T-cell) proliferation, production of interferon gamma (IFNγ), gamma immunoglobulin (IgG), interleukin 12 (IL-12) and interleukin 10 (IL-10) or reduction in shedding of said Chlamydia or said Helicobacter.

16. A method of manufacturing an oral composition, comprising:
mixing a lipid formulation containing at least 30% C₁₆ to C₁₈ fatty acids and at least one antigen from Chlamydia, where said composition is effective for preventing, decreasing or treating a mucosal infection caused by said Chlamydia.

17. The method of claim 16, wherein said at least one antigen is selected from the group consisting of major outer membrane protein (MOMP), a 60 kDa-62 kDa cysteine-rich protein membrane protein, a 15 kDa cysteine-rich membrane protein, a 74 kDa species-specific protein, a 31 kDa eukaryotic cell-binding protein or a 18 kDa eukaryotic cell-binding protein.

18. The method of claim 16, wherein said MOMP is a serotype selected from the group consisting of A, B, Ba, C, D, E, F, G, Hi, I, J, K, L1, L2 or L3.

19. The method of claim 17, wherein said serotype is selected from the group consisting of D, E, F, G, H, I, J, K and L and said method is effective use is to prevent, decrease or treat a genital infection caused by Chlamydia.

20. The method of claim 16, wherein said lipid is selected from the group consisting of lipid C, lipid K, lipid PK and lipid SPK.

21. A method of manufacturing an oral composition comprising:
mixing a lipid formulation containing at least 30% C₁₆ to C₁₈ fatty acids and at least one antigen from Helicobacter, said composition effective-for preventing, decreasing or treating a mucosal infection caused by said Helicobacter.

22. The method of claim 21, wherein said at least one antigen is killed-whole cell Helicobacter pylori.

23. A kit, containing:
A composition of claim 1; and
instructions for use.


SEQUENCE LISTING (3 sequences)

SEQ ID NO: 1 - DNA, 20 bp, Burkholdria pseudomallei
  CpG-ODN 1820
tccatgacgttcctgacgtt

SEQ ID NO: 2 - DNA, 17 bp, Helicobacter pylori
  Helicobacter specific primer Hp 001
tatgacgggtatccggc

SEQ ID NO: 3 - DNA, 19 bp, Helicobacter pylori
  Helicobacter specific primer Hp 002
attccacttacctctccca